(19) United States
(12) Patent Application Publication
(10) Pub. No.: US 2019/0209097 A1
(43) Pub. Date: Jul. 11, 2019

(54) SYSTEM AND METHODS FOR EARLY DIAGNOSIS OF AUTISM SPECTRUM DISORDERS

(71) Applicant: THE GENERAL HOSPITAL CORPORATION, Boston, MA (US)

(72) Inventors: Katherine M. Martien, Dover, MA (US); Joseph R. Isler, New York, NY (US); Martha Herbert, Somerville, MA (US)

(21) Appl. No.: 15/573,852

(22) PCT Filed: May 16, 2016
(86) PCT No.: PCT/US16/32729

§ 371 Date: Nov. 14, 2017

Related U.S. Application Data
(60) Provisional application No. 62/162,011, filed on May 15, 2015.

(51) Int. Cl. A61B 5/00; A61B 5/048; G16H 50/30

(52) U.S. Cl. CPC A61B 5/7246; A61B 5/4064; A61B 5/048; A61B 5/6814; G16H 50/30; A61B 5/725; A61B 5/4076; A61B 5/7282

(57) ABSTRACT

The present disclosure a system and methods for early diagnosis of neurodevelopmental or neurobehavioral diseases, such as autism spectrum disorders (“ASD”). In one aspect, a method for determining a risk for a neonatal patient to develop as ASD is provided. The method includes coupling a sensor assembly comprising plurality of electroencephalogram (“EEG”) sensors to a neonatal patient, and acquiring, using the sensor assembly, EEG data during a sleep state of the neonatal patient. The method also includes analyzing the EEG data to determine neural signatures indicative of a brain activity of the neonatal patient during the sleep state, and generating, based on the neural signatures, a composite representing a neurofunctional profile of the neonatal patient. The method further includes determining a risk for the neonatal patient to develop an autism spectrum disorder (“ASD”) by comparing the composite to a reference, and generating a report indicating the risk.

### CROSS-REFERENCE TO RELATED APPLICATIONS

This application claims the benefit of, and incorporates herein in its entirety, U.S. Provisional Patent Application Ser. No. 62/162,011 filed on May 15, 2015 and entitled “SYSTEMS, DEVICES AND METHODS FOR ANALYZING AUTISM SPECTRUM DISORDERS.”

### STATEMENT REGARDING FEDERALLY SPONSORED RESEARCH

This invention was made with government support under AS073092P3 awarded by U.S. Department of Defense. The government has certain rights in the invention.

### BACKGROUND

The field of the disclosure relates to systems and methods for diagnosing and controlling brain disorders. More particularly, the present disclosure is directed to determining risk and severity of a neurobehavioral disorder, such as Autism Spectrum Disorder (“ASD”), based on early brain activity measurements.

Neurodevelopmental disorders, and more particularly ASDs, are characterized by a wide variety of impairments in social comportment, communication and executive function, often involving inflexibility of interests, and repetitive, stereotypical behavior. Present diagnostic methods for these neurologically-based disorders utilize behavioral, emotional and psychological assessments that can only be performed once behavior is sufficiently well developed to allow for reliable diagnosis. In addition, such diagnosis is not possible until at least the second year of age. With approximately 1 in 100 children being affected by ASD, and incidence appearing to be on the rise, ASDs, and other neurodevelopmental disorders, are becoming increasing health concerns.

The presentation of ASD is highly heterogeneous, and may depend on a number of factors, including genetic and epigenetic factors. Structural brain changes appear to be already present by 12-15 months of age in infants later diagnosed with autism (“LDA”), as evidenced by increased cerebral volume measuring using magnetic resonance imaging (“MRI”). Also, the IBIS Network, based on MRI and diffusion tensor imaging (“DTI”), has shown microstructural abnormalities in white matter tracks and corpus callosum, with increased corpus callosum area and thickness by 6 months of age in IDA. In fact an increasing body of work suggests that structural brain changes are present even before birth. However, despite a number of attempts, there is no universally established MRI-based criteria for diagnosing ASD. Functional brain changes are also being shown at increasingly younger ages. One study showed elevated phase lag alpha connectivity at 14 months in IDA, while another showed reduced intra-hemispheric connectivity by 12 months of age in IDA. Poor eye contact in children with ASD has also motivated studies on eye gaze, with abnormal neural responses to eye gaze shift by 6-10 months of age reported in IDA. Another group reported that visual fixation to eyes, normal at 2 months, has already deteriorated in IDA patients by 6 months of age.

Treatment protocols aimed at improving clinical outcomes, such as the Early Start Denver Model (“ESDM”), have demonstrated improved behavioral outcomes, as well as improved neurophysiologic processing by event-related potential (“ERP”) to faces and objects. Also, trials directed to neuropathologic and metabolic mechanisms in children and adults diagnosed with ASD have shown promise in core features. For instance, Bumetanide, Sulforaphane, Oxytocin and Corticotrophin releasing factor (“CRF”) modulators have been utilized to help improve brain health, function, and later behavioral outcomes.

In light of these studies, and others, initiating treatment before brain function declines may be the key to achieving optimal outcomes. However, challenges remain for providing interventions in newborn infants, especially those aimed to change brain development, due to ethical and practical reasons. For instance, ethical issues around early identification and treatment of infants destined to potentially develop ASDs range from the impact of very early diagnosis on parent-infant bonding, parental stress and parenting more generally, to issues around neurodiversity. Moreover, biomarkers with sufficiently high sensitivity and specificity for early diagnosis have remained elusive both due to disease heterogeneity as well as rapid brain development early in life.

Therefore, there is a great need for systems and methods aimed at early identification of neurodevelopmental diseases, including ASD.

### SUMMARY

The foregoing and other aspects and advantages of the invention will appear from the following description.

In one aspect of the disclosure, a method for determining a risk for a neonatal patient to develop an autism spectrum disorder (“ASD”) is provided. The method includes coupling a sensor assembly comprising plurality of electroencephalogram (“EEG”) sensors to a neonatal patient, and acquiring, using the sensor assembly, EEG data during a sleep state of the neonatal patient. The method also includes analyzing the EEG data to determine neural signatures indicative of a brain activity of the neonatal patient during the sleep state, and generating, based on the neural signatures, a composite representing a neurofunctional profile of the neonatal patient. The method further includes determining a risk for the neonatal patient to develop an autism spectrum disorder (“ASD”) by comparing the composite to a reference, and generating a report indicating the risk.

In another aspect of the disclosure, a method for determining a likelihood for a neonatal patient to develop a neurobehavioral disease is provided. The method includes receiving electroencephalogram (“EEG”) data acquired from a neonatal patient during a sleep state, and generating at least one of a spectral power and coherence information using the EEG data. The method also includes assembling a neurofunctional profile of the neonatal patient using the at least one of spectral power and coherence information, and correlating the neurofunctional profile with a reference to determine a likelihood for the neonatal patient to develop a neurobehavioral disease. The method further includes generating a report using the likelihood.

In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a diagram of an example monitoring system, in accordance with aspects of the present disclosure.

FIG. 1B is a diagram of an example computer-readable media, in accordance with aspects of the present disclosure.

FIG. 2 is a flowchart setting forth steps of a process, in accordance with aspects of the present disclosure.

FIG. 3 is another flowchart setting forth steps of a process, in accordance with aspects of the present disclosure.

FIG. 4 shows graphs comparing electroencephalogram (“EEG”) power for low risk and high-risk patients during sleep.

FIG. 5 shows graphs comparing hemispherical EEG power for low risk a high-risk patients during sleep.

FIG. 6 shows graphs comparing integrated hemispherical EEG power for low risk and high-risk patients during sleep.

FIG. 7 shows graphs comparing EEG coherence for low risk and high-risk patients during sleep.

FIG. 8A is a graphical illustration showing correlations of EEG coherence with Best Estimate Diagnostic score for high-risk patients.

FIG. 8B is another graphical illustration showing correlations of left hemisphere EEG power with Best Estimate Diagnostic score for high-risk patients.

FIG. 8C is yet another graphical illustration showing correlations of right hemisphere EEG power with Best Estimate Diagnostic score for high-risk patients.

FIG. 9A is a graphical illustration showing correlations of EEG coherence with Autism Diagnostic Observation Schedule for high-risk patients.

FIG. 9B is another graphical illustration showing correlations of left hemisphere EEG power with Autism Diagnostic Observation Schedule for high-risk patients.

FIG. 9C is yet another graphical illustration showing correlations of right hemisphere EEG power with Autism Diagnostic Observation Schedule for high-risk

FIG. 10A is a graphical illustration showing correlations of EEG coherence with Pervasive Developmental Disorder-Behavioral Index for high-risk patients.

FIG. 10B is another graphical illustration showing correlations of left hemisphere EEG power with Pervasive Developmental Disorder-Behavioral Index for high-risk patients.

FIG. 10C is yet another graphical illustration showing correlations of right hemisphere EEG power with Pervasive Developmental Disorder-Behavioral Index for high-risk patients.

FIG. 11A is a graphical illustration showing correlations of EEG coherence with Mullen score for high-risk patients.

FIG. 11B is another graphical illustration showing correlations of left hemisphere EEG power with Mullen score for high-risk patients.

FIG. 11C is yet another graphical illustration showing correlations of right hemisphere EEG power with Mullen score for high-risk.

FIG. 12A is a graphical illustration showing correlations of EEG coherence with Vineland score for high-risk patients.

FIG. 12B is another graphical illustration showing correlations of left hemisphere EEG power with Vineland score for high-risk patients.

FIG. 12C is yet another graphical illustration showing correlations of right hemisphere EEG power with Vineland score for high-risk patients.

### DETAILED DESCRIPTION

The lack of early biomarkers for identifying neurodevelopmental or neurobehavioral disorders, such as autism spectrum disorders (“ASDs”), remains an ongoing clinical challenge. With apparently rapidly increasing occurrences and devastating psychological, sensory-perceptual, motor, socio-emotional and behavioral manifestations, neurodevelop mental diseases will have significant social and economic costs. In particular, autism and related disorders are rarely associated with early death, and so a long-term heavy burden falls on families and communities, as well as societies and economies.

A number of studies point to genetic predisposition being responsible, to varying degrees, for the development of many neurodevelopmental disorders. Also there is strong evidence that environmental factors and “gene x environment” interactions are contributing to this epidemic. While curbing the genetic and environmental phenomena is of paramount importance, it is likely that the needed environmental changes will transpire slowly. On the other hand, while genetic interventions may potentially appear sooner, measures of the efficacy of such treatments would still be required.

While many in the public and even in the medical and psychological/psychiatric professions consider ASDs to be untreatable lifelong conditions, there is a growing body of data demonstrating that improvements, remissions and even loss of diagnosis are achievable. This includes outcome data from various behavioral, medical and lifestyle interventions, as well as individual cases of autism recovery that have recently been studied. Hence, early detection of neurodevelopmental or neurobehavioral disorders is highly desirable. In fact, since critical brain development occurs within the first year of life, detection in the first year of life, and as early as the neonatal period, within approximately a month from birth, may be most beneficial.

Therefore, the present disclosure describes a system and methods for early identification of neurodevelopmental disorders. In particular, the present approach includes analysis of electroencephalogram (“EEG”) data, and potentially other data, acquired from a neonatal patient during a neonatal period. Based on determined neural states determined from the data, a composite representing a neurofunctional profile of the neonatal patient can be obtained and compared to a reference in order to determine a risk or likelihood of developing a neurobehavioral disease, such as ASD, later in life. In some aspects, spectral power and coherence information, as well as other information generated from the data, may be used to determine neural states indicative of the neurofunctional profile of the neonatal patient and risk for neurobehavioral or neurodevelopmental disorders. In this manner, previously undiagnosed patients can be identified early in life.

Because the present approach can achieve discrimination between brain-disrupted states associated with disease, from non-disrupted state, it allows for vulnerable individuals to be identified and for intervention far earlier in brain development than is currently possible. As appreciated, this can lead to potential improvements in the quality and coherence of the brain functional development, as well as processes that underlie social/emotional, behavioral, sensory processing, motor, attentional and cognitive development. Such extremely early intervention may even eventuate in the prevention of the development of an autism spectrum or related disorder.

As will be described, the present approach is based upon the discovery by the inventors that neurophysiological patterns or neural signatures measured using EEGs during a neonatal period can be indicative of behavioral, and other abnormalities later in life. In fact, given the drastic brain changes that occur during the first years of life, thus far it has been unclear whether any early biomarkers for determining risk later in life exist. As such, present diagnostic methods typically use behavioral, emotional and psychological assessments that are performed once behavior is sufficiently well developed, which is typically after the age of two. However, as may be appreciated, the best opportunities for preventative or ameliorative intervention can be missed by that time.

Although the present disclosure includes description related to Autism and Autism Spectrum Disorders (“ASDs”), it may be readily appreciated that the present system and methods can be applied to a variety of neurodevelopmental diseases, including Language Disorders, Sensory Integration Disorders, Motor Disorders (i.e. fine and gross motor dyspraxia), certain types of Cognition, Attention, Learning and Memory Disorders, and potentially later onset Neuropsychiatric, Neurobehavioral and Neurointegrative Disorders, and others.

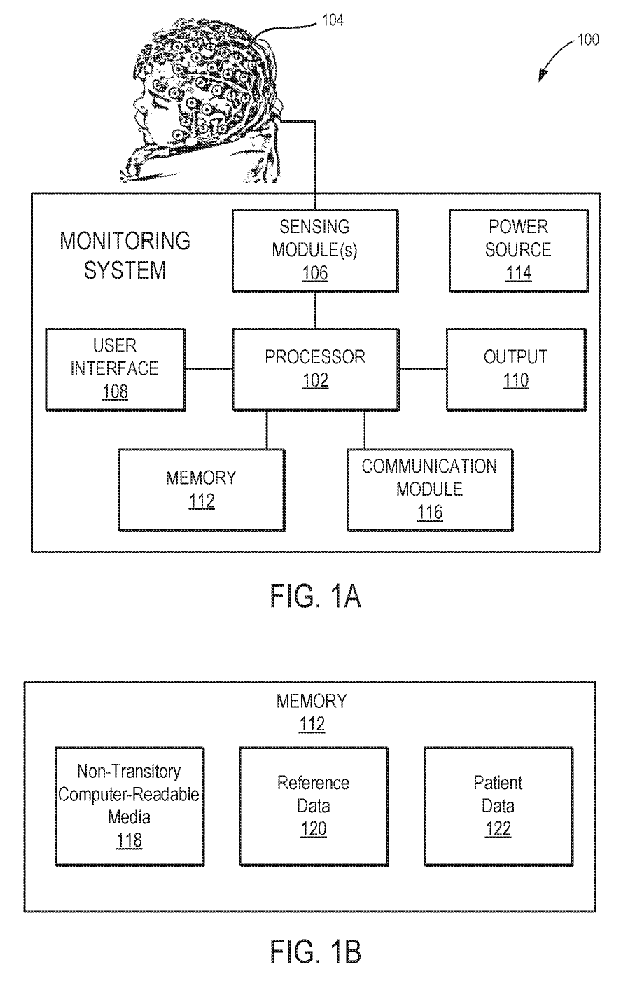

Turning now to FIG. 1A, a diagram of an example monitoring system 100, in accordance with aspects of the present disclosure, is shown. In general, the monitoring system 100 may include a processor 102, and a sensor assembly 104 coupled to one or more sensing modules 106. The monitoring system 100 also includes a user interface 108, an output 110, a memory 112, and a power source 114. In some implementations, the monitoring system 100 can be a computer, workstation, a network server, a mainframe or any other general-purpose or application-specific computing device. The monitoring system 100 may also be a portable device, such as a mobile phone, laptop, tablet, personal digital assistant (“PDA”), multimedia device, or any other portable device.

As shown in FIG. 1A, the sensor assembly 104 may include an electroencephalogram array (“EEG”), consisting of multiple EEG sensors arranged at various locations about a patient's head, for example using a 10-20 lead system. In one embodiment, the sensor assembly 104 includes at least 4 leads, each two-lead pair being configured to measure a brain hemisphere. By way of example, the leads may be arranged at locations associated with C3, C4, O1 and O2. It may be readily appreciated that other lead configurations may also be possible.

In addition, it is contemplated that the system 100 may include a number of other biosensors, integrated into or separate from the sensor assembly 104. In particular, monitoring system 100 may also include mechanisms or sensors for detecting galvanic skin response (“GSR”) to measure arousal to external or internal stimuli. The monitoring system 100 may also include cardiovascular sensors, including electrocardiographic and blood pressure sensors, and also ocular microtremor sensors. One realization of the sensor assembly 104 Laplacian EEG electrode layout with additional electrodes to measure GSR, ocular microtremor, and others. Another realization may incorporate an array of electrodes that could be combined in post-processing to obtain any combination of electrodes found to optimally detect the neural signatures. Another realization may utilize a high-density layout sampling the entire scalp surface of a patient using between 64 to 256 sensors for the purpose of source localization.

The sensing module(s) 106 may be connected to the sensor assembly 104 via a wired or wireless connection and include a variety of capabilities. For instance, in some embodiments, the sensing module(s) 106 may include capabilities for detecting and filtering noise with a specific noise profile from measured biosignals. The sensing module(s) 106 may also include capabilities for amplifying measured biosignals, as well as converting filtered and/or amplified signals from an analog to a digital form to be processed further by the processor 102. In some implementations, amplification may be performed or included into the sensor assembly 104, or sensors or electrodes therein. In addition, the sensing module(s) 106 may also include capabilities for sensing impedance, identifying, for example, defective leads, or when the sensor assembly 104 has been coupled or decoupled from a patient.

In addition to being configured to carry out steps for operating the monitoring system 100, the processor 102 may be further configured to determine a risk or likelihood for a patient to develop a neurobehavioral or neurovelopmental disorder, such as ASD. In some aspects, the processor 102 may be configured to retrieve and analyze EEG data acquired from a neonatal patient during sleep to determine neural signatures indicative of brain activity. In the analysis, a wide variety of data or information may be generated, including spectral information, power information, coherence information, phase information, synchrony information, asymmetry information, and so forth. For example, hemispherical power spectra and coherence, and other parameters, may be computed by the processor 102 using acquired EEG data. Using such information, neural signatures, in the form of various signal amplitudes, phases, frequencies, power spectra, coherences, cross-frequency couplings, synchrony, symmetry, and so forth, may be assembled.

By way of example, a neural signature based on computed power spectra may indicate measured absolute, or relative power at various frequencies and locations about the patient's head. Also, a neural signature may reflect differences or symmetries in spectral power between different locations about the patients head, such as between different brain hemispheres.

In some aspects, a composite representing a neurofunctional profile of the monitored patient may be generated by the processor 104 based on a number of determined neural signatures. The composite may be in the form of a weighted combination of various neural signatures extracted from the acquired data, based, for instance, on their sensitivity or specificity for distinguishing the risk of developing a neurobehavioral or neurovelopmental disorder. For example, a composite may include spectral power and coherence information.

The processor 104 may then utilize neural signatures or an assembled composite to determine a risk for a patient to develop a neurobehavioral or neurovelopmental disorder, such as ASD, by performing a comparison or correlating the composite with a reference. In some embodiments, the reference may be in the form of a listing or database that includes information from a population of age-matched neonates or infants. In other embodiments, the reference may include baseline, or other data or information from the same patient. For, the reference may include hemispheric coherence values either at low or high frequencies, as well as other values. In this case, a statistical result can be obtained by comparing the coherence values at low and high frequencies in order to generate a statistical result that parses ASD from non-ASD patients, for example.

Therefore, the processor 104 may perform a statistical analysis to produce a statistical prediction or compute a likelihood quantifying a risk of the patient to develop a neurobehavioral or neurovelopmental disorder, and then generate a report indicating the risk provided via the output 110. In determining the risk, the processor 102 may take into consideration a variety of other information in addition to measured neural signatures or composite, including one or more patient characteristics, such as chronological age, post-menarchal age, sex, medical condition or history, genetic and other risk factors, and so forth.

The monitoring system 100 may operate as part of, or in collaboration with, one or more computers, devices, machines, mainframes, servers, cloud, the internet, and the like. As such, the monitoring system 100 also includes a communication module 116 that is configured to not only enable communication among the processor 102, sensing modules 106, user interface 108, output 110 and memory 112, but also communication with external systems.

As described, the monitoring system 100 of FIG. 1A includes a memory 112 accessible by the processor 102 that can include a variety of data and information. Alternatively, or additionally, the processor 102 may access an external database, server, or other data storage location (not shown). As shown in FIG. 1B, in some embodiments, the memory 112 may include non-transitory computer readable media 118, reference data 120, patient data 122. In some aspects, the non-transitory computer readable media 118 may include instructions for acquiring, or accessing EEG data acquired from a patient, and processing the data to determine a risk or likelihood of a patient to develop a neurobehavioral or neurovelopmental disorder, in accordance with aspects of the present disclosure.

The reference data 118 may be in the form of reference listings or look-up tables that include patient categories, such as various age categories, risk categories, and other categories, along with associated signals, signal markers or neural signatures. For example, signals, signal markers or neural signatures can include various signal amplitudes, phases, frequencies, power spectra, spectrograms, coherograms, and so forth, associated with high or low risk for developing a neurobehavioral or neurovelopmental disorder, such as ASD. The patient data 120 may include a wide variety of information or parameters accessed from a storage location or entered via the user interface 108. These may include age, sex, head circumference, medical conditions, medical ID, baseline parameter values, genetic predispositions, prior analysis results, and so forth.

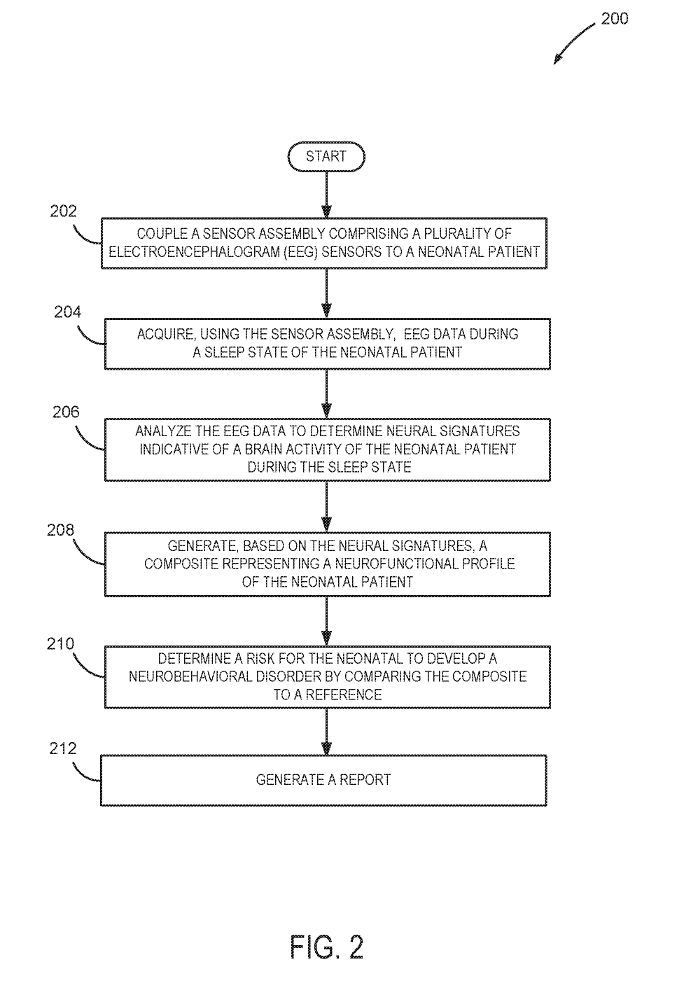

Turning now to FIG. 2, a flowchart setting forth steps of process 200 in accordance with aspects of the present disclosure is shown. The process 200 may begin at process block 202 with coupling a sensor assembly, as described with reference to FIG. 1A to a neonatal patient. In some aspects, a technician may verify information associated with the subject, including any health concern, and so forth. The technician may also note in a patient record a time and data, a patient's name, ID number, date and time of birth, EDC, sex, head circumference, and so forth. As described, in some implementations, the sensor assembly may include a minimum of 4 derivations in the ILEA 10-20 system, including C3, C4, O1 and O2, although other electrode locations may also be possible.

Then EEG data may be acquired using the sensor assembly during a sleep state of the neonatal patient, as indicated by process block 204. In some aspects, signals from the sensors in the sensor assembly may be tested to assure that impedances are below a threshold and signals are free from artifacts. Alternatively, a processor may initiate a test phase to automatically test these. In addition, data acquisition need not be limited to sleep states, and may be performed during a resting or awake states. Also, data acquisition may be performed while stimulating the patient.

In order to determine a sleep state of the patient, a technician may perform an observation for behavioral signs of sleep, such as closed eyes, no voluntary movements, breathing slow and sustained for 2 minutes for instance. Alternatively, a processor may determine a sleep state of the patient using EEG, respiratory, and other data. Acquisition of the EEG data may be continued until a sufficient amount of data is obtained. For example, EEG data may be recorded over at least a 10-minute sleep period, although other sleep periods may also be utilized.

Then, at process block 206, EEG and other data may be analyzed to determine neural signatures indicative of brain activity of the patient. As described, in some aspects, spectral power and coherence information may be generated in the analysis. For example, a power spectrum for left and/or right hemispheres may be obtained by applying Fast Fourier Analysis techniques. Also, a coherence between signals from the left and right hemisphere or within hemispheres may also be computed. Using such information, neural signatures, in the form of various signal amplitudes, phases, frequencies, power spectra, coherences, cross-frequency couplings, synchrony, symmetry, and so forth, may be assembled.

As indicated by process block 208, a composite representing a neurofunctional profile of patient may be generated based on the determined signatures. As described, the composite may include spectral power, coherence, and other information, and may be in the form of a weighted combination of various neural signatures extracted from the acquired data, based, for instance, on their sensitivity in distinguishing the risk of developing a neurobehavioral or neurovelopmental disorder.

The composite may then be used at process block 210 to a risk or likelihood for the patient to the develop a neurobehavioral or neurodevelopmental disorder. In some aspects, a statistical analysis may be performed at process block 210. In particular, the composite may be compared to a reference that categorizes neurofunctional profiles according to age, sex, disorder, risk factors, and so forth. For example, a greater hemispheric asymmetry in low frequency power, and reduced or absent low frequency interhemispheric coherence, may be compared to age matched controls at process block 210.

In some aspects, the composite may be compared to a reference that includes an intra-patient standard at process block 210. For example coherence values at different frequencies may be compared. Specifically, this relies upon the finding by the inventors that at higher frequencies EEG synchrony is reflective of volume conduction bias in the EEG (i.e. not reflective of true neural synchrony between regions), and can be considered as a null level within each patient. Thus a within-patient measure of synchrony may include whether or not there is significantly higher coherence at lower frequencies compared to a mean of the coherence spectrum over higher frequencies. By way of example, low or lower frequencies can be in a range less than approximately 6 Hz, while high or higher frequencies can be in a range approximately greater than 16 Hz. It may be appreciated that other frequencies ranges may be utilized as well.

These and other measures, may be combined into a composite score that weights them according to their sensitivity in distinguishing high risk from low risk. The composite score may then be used to assess risk in a neonate patient for developing a neurobehavioral disorder. For example, the composite score may be compared to a threshold, or different ranges of values, to determine the risk.

A report may then be generated, as indicated by process block 212. The report may include a variety of information, and be provided in the form of a printed, electronic or real-time display. The report may include raw or processed data, waveforms, neural signature information, indications of a risk or likelihood of developing a neurodevelopmental or neurobehavioral disorder, as well as information related to patient-specific characteristics, including patient age, sex, medical condition, ID, and so forth. In some aspects, the report may be stored in the patient's medical record.

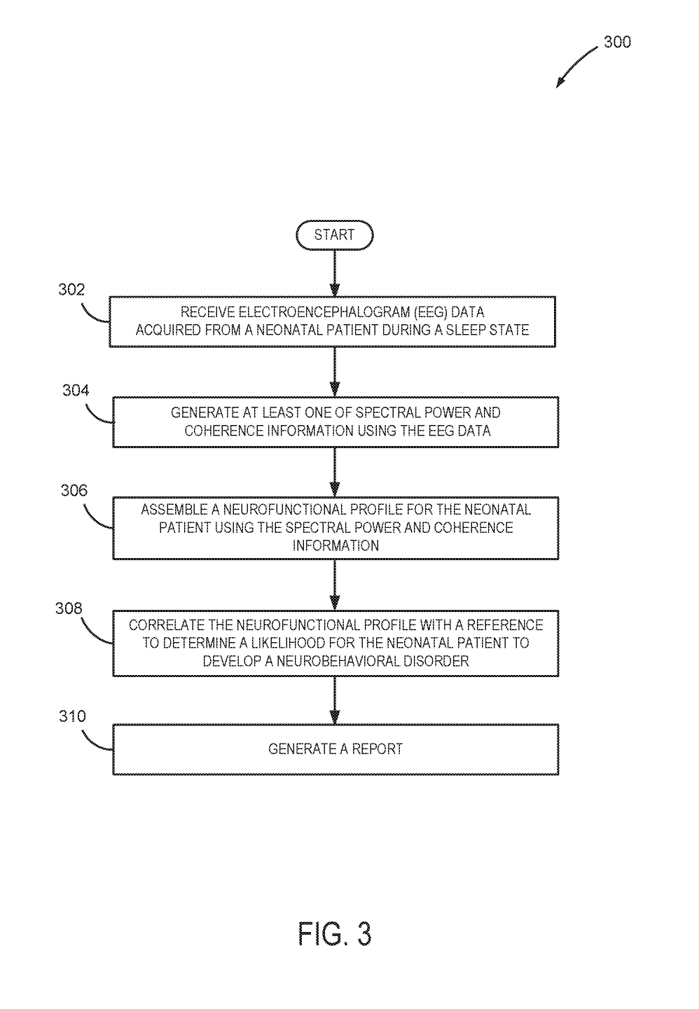

Turning now to FIG. 3, a flowchart setting forth steps of another process 300 in accordance with aspects of the present disclosure is shown. The process 300 may begin at process block 302 with receiving EEG and other data acquired from a neonatal patient during a sleep state. Using the EEG data, at least one of spectral power and coherence information is generated, as indicated by process block 304. Other information may also be generated at process block 304, including phase information, synchrony information, symmetry information, and so forth.

Such information may then be used at process block 306 to assemble a neurofunctional profile for the patient, for instance, in the form of a composite. As described, in some aspects, the composite may derive from a combination of different neural signatures, each weighted in accordance with their sensitivity or specificity for distinguishing the risk of developing a neurodevelopmental or neurobehavioral disorder.

The neurofunctional profile may then be correlated with a reference to determine a likelihood for the patient to develop a neurodevelopmental or neurobehavioral disorder. In some aspects, a coherence between a right brain hemisphere and a left brain hemisphere of the neonatal patient, or portions thereof. That is, overall hemispherical coherence values as well as coherence values between specific contralateral and ipsilateral regions may be performed to determine the likelihood. In other aspects, the likelihood determined by comparing coherence values at multiple frequencies for various locations about the neonatal patient's head. For example, coherence values at low frequencies (i.e. less than approximately 6 Hz) may be compared with coherence values at high frequencies (i.e. less than approximately 16 Hz). A report may then be generated, as indicated by process block 310.

In addition to descriptions above, specific examples are provided below, in accordance with the present disclosure. These examples are offered for illustrative purposes only, and are not intended to limit the scope of the present invention in any way. Indeed, various modifications in addition to those shown and described herein will become apparent to those skilled in the art from the foregoing description and the following example and fall within the scope of the appended claims.

### Example

In the present study, EEGs were recorded in the neonatal period in a cohort of infants at high-risk for developing as ASD by virtue of being the infant sibling of a child with an ASD. The cohort was identified prenatally, studied at 42 weeks PMA and followed prospectively. Outcome assessments were carried out at 20 and 30 months of age with additional assessments if a regression was suspected. Spectral analyses of the neonatal EEGs in the high-risk infants were compared to that of a identified low-risk infant cohort (CHIME study) seeking evidence of neurophysiologic differences in the neonatal period. High-risk infant EEG Spectral data were further analyzed seeking neonatal neurophysiologic correlates of developmental outcome, e.g. ASD diagnostic classification and severity of ASD behaviors.

### Methods

### Approvals

This study was conducted as a part of a larger study funded by the US Department of Defense entitled: A Prospective Multi-System Evaluation of Infants at Risk for Autism. Within the larger study, the present comparative and prospective correlational study was conducted using EEG recorded in the newborn period in infants at high-risk for developing as ASD. Neonatal EEGs in high-risk infants were compared to a normative age-matched cohort. High-risk infants were followed into the second and third years of life years when formal outcome assessments of behavior and cognitive development were performed. Behavioral and developmental outcome data were analyzed for correlations with newborn EEG characteristics to seek a neonatal EEG biomarker predictive of a high-risk of developing autistic behavior in early childhood. The study was carried out at the Massachusetts General Hospital LADDERS Clinic/Lurie Center in the TRANSCEND Research Program and the rationale, study design, methods, recruiting materials and consents were approved by the Internal Review Boards of both the Massachusetts General Hospital and the United States Department of Defense. Data analysis was carried out at MGH and at Columbia Presbyterian Medical Center.

### Study Design

A prospective study was performed for infants at high-risk of developing as autism spectrum disorder by virtue of having an affected older sibling. At risk infants were identified prenatally, followed through birth and underwent a high density EEG protocol soon after birth aiming at 42 weeks gestational age. Outcome assessments were carried out at 20 and 30 month of age to determine developmental outcome. Neonatal EEG power and coherence in the HRA infants and in an age-matched, normative low risk (LRA) sample were analyzed seeking evidence of neural signatures that would discriminate the two groups. Neonatal EEG power and coherence in HRA infants were then examined relative to the outcome data seeking correlations between neonatal neural signatures and behavioral manifestations at outcome time points.

### Subject Recruitment, Inclusion and Exclusion Criteria

In order to develop a cohort of newborn infants at high-risk of developing an ASD (HRA), biological mothers of children carrying a clinical diagnosis of an ASD (Probands) who were pregnant with an infant sibling of same parentage and, therefore, at high-risk of developing an ASD (HRA), were recruited from the clinic population and from the community at large. Inclusion criteria required the pregnant mothers of probands (MOP) to be generally healthy, free of obstetrical complications at enrollment and free of know genetic or chronic medical conditions. Probands were required to be free of known genetic, metabolic or other disorder which in the opinion of a PI might have induced their ASD in a fashion that would not be associated with increased risk of recurrence in the infant sibling, i.e. congenital rubella. The diagnosis of ASD in the clinically-diagnosed probands was confirmed by SCQ, ADOS and/or ADI-R performed by research reliable testers. As outcome measures were in English, participating families were required to be using English as the principal language spoken in the home (defined by >50%).

The pregnant mothers were followed prospectively through delivery. HRA infants were recruited soon after birth into the study if they met inclusion criteria: 1) free of significant complications during maternal pregnancy, labor and delivery, 2) delivery at 36 weeks GA or later, 3) normal APGAR score and free of intra-uterine growth retardation, microcephaly, pre-, peri- or postnatal events associated with potential brain injury and other medical conditions which in the opinion of the PI impacted the infant's development or neurologic wellbeing. Data collection in HRA infants included: Gestational Age (GA) at birth, Post Menstrual Age (PMA), Chronological Age (CA) at EEG and gender.

### EEG Data Collection

The HRA infants underwent high density EEG at ˜42 weeks PMA. Upon arrival for the EEG, well-being was confirmed by history and physical, the infants were nursed/fed and then held by a caregiver while a 128-electrode HydroCel Geodesic Sensor Net sized to head circumference and pre-soaked in KCl, was placed on the infant's head by trained research staff. Impedance of 50 kOhms or less was documented in all leads at outset. EEG was recorded in an acoustically and RF shielded room on an EGI 128 channel, Geodesic EEG System 250 using Net Station with the infant lying supine at approximately 45 degrees in an infant seat with a parent and study staff in attendance. “Sleeping” state (SS) was established by a trained observer based on the HRA infant being in an eyes closed, motionless state with regular, slowed breathing for at least 2 minutes before the sleep session was initiated and through the continuous SS recording session. A minimum of ten minutes of sustained sleep state was recorded for analysis. A formal polysomnogram was not performed, thus sleep state (SS) was not parsed into active (AS) or quiet (QS) (see normative data set below). EEG data was stored on discs and saved for analysis, which was performed after outcome assessments were completed, e.g. testers were blind to EEG results.

### Outcome Assessment

HRA subjects were followed prospectively at regular intervals over the next two and a half years with formal outcome cognitive and behavioral assessments performed at 20 and 30 months of age. Parents were instructed to watch for regression and return for an additional assessment if regression was suspected clinically. The outcome battery consisted of two direct assessments of the child and two parent report measures, and included medical history and physical exam, Autism Diagnostic Observation Schedule (ADOS), including a Calibrated Severity Score (CSS), Mullen Scales of early Learning (MSEL) including subdomains and composites, Pervasive Developmental Disorder-Behavioral Index (PDD-BI), including sub-domains, composites and Social Discrepancy (SOCDSC), and Vineland Adaptive Behavioral Scales (VAGS) subdomains and composites.

ADOS (CSS) and PDD-BI (AUC and SOCDSC) each generated outcome classifications, and were used in combination to establish Best Estimate Diagnostic Classification: Autism (A), Autism Spectrum Disorder (ASD), Broader Autism Phenotype (BAP), and Non-spectrum (NS). The ADOS CSS and PDD-BI were further used to generate a severity profiles of spectrum diagnosis and related behaviors. The Mullen Scales of Early Learning (MSEL) and Vineland Adaptive Behavior scales (VABS) were used to establish a broader developmental profile. All subject data was placed in the research program's account in the REDCap research database maintained by Harvard Medical School.

### Data Analysis

### EEG Data

Raw EEG data from HRA infants at 42 weeks PMA was reviewed for quality and cleaned of artifact and prepared for analysis in comparison to a normative EEG sample deemed at no know risk and therefore Low risk for ASD (LRA). HRA infant EEG was recorded in a session that included multiple experimental paradigms designed to elicit event related potentials in waking and sleep states. Here, all analyses were restricted to a 10-minute baseline period. After recording, HRA infant EEG data were filtered for line noise with a 1600 point linear phase (FIR) software notch filter (4 Hz wide notches with 60 dB falloff within 2 Hz of the notch edges) for 60 Hz and its harmonics up to 360 Hz. For each 30 s period within the baseline, power and coherence were computed using the Welch method with up to 30 1 s fast Fourier transforms. Prior to averaging cross-spectra, each second was quantitatively examined for head movement and other artifacts.

For each electrode, data were rejected if any of the following criteria were met: absolute sample-to-sample change was greater than 25 pV; absolute value was greater than 100 pV; standard deviation was greater than 40 pV; and spectral slope between 20 and 200 Hz was greater than −0.1 (to detect muscle artifact). Seconds with more than 16% of electrodes matching one of the above criteria were rejected. For the remaining seconds, eye movements were detected using a bipolar montage of smoothed (40-point boxcar) raw data from 11 channels near the eyes and seconds with suspected eye movements were rejected (criteria were four or more good bipolar channels with an absolute first derivative greater than 0.5 μV per sample). An extremely conservative was taken in this regard, removing seconds even with only weak evidence of eye movements. After the above preprocessing, data were referenced to the average over all good electrodes at each time point in order to characterize topography in an unbiased fashion (Junghofer, Elbert, Tucker & Braun, 1999). To match EEG data in the LRC cohort (see below), further analysis was restricted to two bipolar derivations, C4-O2 and C3-O1 in the international 10-20 system, permitting statistical analysis of differences in power and coherence comparing the low- vs. high-risk cohorts. If fewer than 10 s remained after all preprocessing, a 30 s epoch was excluded.

Power and coherence were then computed using the average cross-spectrum over the remaining seconds. In order to source a cohort of infants at low of autism (LRC), a normative, open source EEG data set generated as a part of the NICHD funded Collaborative Home Infant Monitoring Evaluation (CHIME) Study was used [Crowell 2002]. Subject data included sex, GA at birth, PMA and CA at monitoring allowing LRC CHIME infants to be selected from the database to generate an age-match LRA cohort for comparison to the 42-week PMA HRA cohort EEGs. An overnight polysomnographic recording was obtained for each CHIME subject at an average postnatal age (PNA) of 4.2±0.78 (SD) weeks. The IPSG included EEG data from central (C3, C4) and occipital (O1, O2) electrode sites recorded with contralateral mastoid references (A1, A2). The CHIME acquisition hardware system (ALICE3) filtered the EEG data from 1 to 40 Hz with a notch filter at 60 Hz, and then digitized the signals with 8 bits per sample at the rate of 100 Hz. For a subset of subject population, CHIME investigators coded sleep state in 30-s epochs from autonomic data in the IPSG. For comparison to the HRA EEG data and to remove a potential for bias from the contralateral mastoid reference, bipolar derivations were formed from the original 4 EEG channels: C3 minus O1 on the left hemisphere and C4 minus O2 on the right hemisphere. All preprocessing and spectral computations were performed as in the HRA cohort, except detection of eye movements. EEG power was log transformed. For comparisons of total power, log power spectra were integrated from 2 to 20 Hz using trapezoidal numerical integrations. All comparisons between groups were tested for significance with unpaired t-tests. All comparisons within groups were tested for significance with paired t tests.

Group power and coherence results for HRA infants was then regressed against group outcome measures seeking correlations between neonatal EEG findings and developmental/behavioral outcome at 30 months: L power, R power and L-R coherence vs. diagnostic classification and Severity of outcome behaviors, e.g. ADOS CSS, PDD-BI sub-domains scores, PDD-BI domain composites, the Autism Composite (AUC) and one discrepancy score (SOCDSC) and the Best Estimate Diagnostic Classification (DxCL) HRA individual power and coherence spectra were compared to LRA group results seeking different patterns of neurophysiologic profiles in the newborn period as revealed by Diagnosis, presence or absence of a regression (delta of ADOS CSS 20-30 mo) and gender. All analyses were performed using the Matlab programming language.

### Results

Subjects—

HRA cohort: Twelve pregnant mothers (MOA), each with at least one living child (Proband) carrying a DSM IV diagnosis of an ASD (Autism or PDD-NOS) confirmed on ADI-R and/or ADOS were enrolled as MOA-Proband pairs into the present study. All MOA and probands met inclusion and exclusion criteria, including maternal report of same parentage for the autistic child and their expected newborn infant. MOA provided consent for themselves and their autistic child. One mother had two probands meeting ASD study criteria. One mother was subsequently determined to be carrying a fetus with Trisomy 18 and exited the study. One mother was diagnosed with fraternal twins and remained in the study.

TABLE 1
Proband Cohort-Best Est. Dx Classification based on ADOS/ADI_R

| Proband for | Proband | Proband | Confirmed | ADOS/A | |
|---|---|---|---|---|---|
| Subject # | Gender | Clin Dx | by | DI Dx | Dx CL |
| 1 | M | Autism | ADOS | ASD | ASD |
| 2 & 5* | M | Autism | ADOS | Autism | Autism |
| 2 &5* | M | PDD-NOS | ADOS | ASD | ASD |
| 3 | M | PDD-NOS | Autism | Autism | Autism |
| 4 | M | PDD-NOS | Autism | Autism | Autism |
| 6 | F | Autism | ADOS | Autism | Autism |
| | | | ADI& | | |
| 7 | M | PDD-NOS | ADOS | ASD | ASD |
| 8 | M | PDD-NOS | ADI | ASD | ASD |
| 9 | M | PDD-NOS | ADOS | Autism | Autism |
| 10 | M | PDD-NOS | ADOS | Autism | Autism |
| 11 | M | Autism | ADOS | Autism | Autism |

*Same 2 probands for the two fraternal twins.

The remaining eleven MOA and their twelve children meeting criteria for autism formed the MOA-Proband pairs cohort. The mother with two probands was also the mother carrying twins. All MOA continued free of obstetrical complications to term and all delivered healthy infants, including the pair of twins. The twelve newborn infant siblings of autistic probands all met study criteria and were enrolled with parental consent soon after birth providing for a cohort of twelve infants at high-risk of developing an ASD (HRA). All twelve infants underwent high density electroencephalograms (HD EEGs) between 41-44 weeks post-menstrual age (PMA), (mean 42.36 weeks). Sleep EEG data was later deemed insufficient for analysis in one female infant. Thus the HRA study cohort consisted of eleven newborn infants, four females and seven males, and included one pair of fraternal twins (Table 1: Proband Cohort, Table 2: HRA cohort).

TABLE 2
HRA Cohort

| | | Gest | Gest | |
|---|---|---|---|---|
| | | Age | Age | Chron |
| | | @ | @ | Age @ |
| Subject | | Birth | EEG | EEG |
| # | Gender | (wks) | (wks) | (wks) |
| 1 | F | 39.57 | 41.86 | 2.29 |
| 2 | F | 38.43 | 42.29 | 3.86 |
| 3 | F | 39.29 | 42.14 | 2.86 |
| 4 | F | 37.43 | 42.14 | 4.71 |
| 5 | M | 38.43 | 42.29 | 3.86 |
| 6 | M | 38.29 | 42.14 | 3.86 |
| 7 | M | 41.14 | 43.57 | 2.43 |
| 8 | M | 39.71 | 42.14 | 2.43 |
| 9 | M | 39.43 | 42.14 | 2.71 |
| 10 | M | 40.00 | 42.43 | 2.43 |
| 11 | M | 39.29 | 42.86 | 3.57 |
| Means | | 39.18 | 42.36 | 3.18 |

### Normative Controls: LRC “CHIME” Cohort

Twenty-three healthy infants from the CHIME dataset with no known risk of autism were identified to have neonatal EEG data that were well age-matched to the HRA cohort (mean GA at birth 39.2 vs 39.3 weeks; mean PMA at EEG 42.4 vs 43.5 weeks) and formed the LRC cohort. [Table 3a]

TABLE 3a
LRC Cohort

| Subject | | Gest Age @ | Gest Age @ | Chro Age @ |
|---|---|---|---|---|
| # | Gender | Birth (wks) | EEG (wks) | EEG (wks) |
| 1 | M | 38 | 42 | 4 |
| 2 | F | 38 | 43 | 5 |
| 3 | M | 41 | 44 | 3 |
| 4 | M | 40 | 44 | 4 |
| 5 | M | 40 | 44 | 4 |
| 6 | F | 38 | 44 | 6 |
| 7 | M | 39 | 42 | 3 |
| 8 | M | 39 | 43 | 4 |
| 9 | M | 39 | 43 | 4 |
| 10 | M | 39 | 44 | 5 |
| 11 | F | 40 | 44 | 4 |
| 12 | F | 39 | 44 | 5 |
| 13 | M | 40 | 43 | 3 |
| 14 | F | 39 | 44 | 5 |
| 15 | F | 38 | 42 | 4 |
| 16 | M | 40 | 44 | 4 |
| 17 | M | 40 | 44 | 4 |
| 18 | M | 39 | 43 | 4 |
| 19 | M | 39 | 44 | 5 |
| 20 | M | 39 | 43 | 4 |
| 21 | M | 39 | 44 | 5 |
| 22 | M | 41 | 44 | 3 |
| 23 | M | 40 | 44 | 4 |
| Mean | | 39.3 | 43.5 | 4.2 |

TABLE 3b
Example within-subject composite

| Measure | LRC p value | HRA p value |
|---|---|---|
| 2 Hz vs mean HF Coh | 2.235E−08 | 0.7405 |
| 4 Hz vs mean HF Coh | 0.0023 | 0.6718 |
| 2 Hz Pwr Asymm | 0.8776 | 0.0073 |
| 4 Hz Pwr Asymm | 0.9875 | 0.0187 |

TABLE 4a
HRA Cohort with 30-month outcome

| | | ADOS | PDDBI | Mullen | Vineland |
|---|---|---|---|---|---|
| | | CA@ | CA@ | CA@ | CA@ |
| Subject | | Outcome | Outcome | Outcome | Outcome |
| # | Gender | (wks) | (wks) | (wks) | (wks) |
| 1 | F | 29.68 | 31.76 | 29.68 | 29.68 |
| 2* | F | 30.18 | 30.54 | 30.18 | 30.14 |
| 3* | M | 30.18 | 30.54 | 30.18 | 30.14 |
| 4 | M | 29.72 | 29.72 | 29.72 | 29.72 |
| 5 | M | 30.24 | 30.21 | 30.24 | 30.24 |
| 6 | F | 33.73 | 33.73 | 33.73 | dnc |
| 7 | M | 30.64 | 30.64 | 30.64 | 32.41 |
| 8 | M | 37.21 | 28.76 | 28.76 | 29.09 |
| 9 | M | 29.91 | 29.91 | 29.91 | 30.34 |
| Mean ages | | 31.28 | 30.65 | 30.34 | 30.22 |

*fraternal twins.

### I. Neonatal EEG Findings:

Neonatal EEG Findings: HRA Vs. LRC Power—Group Data

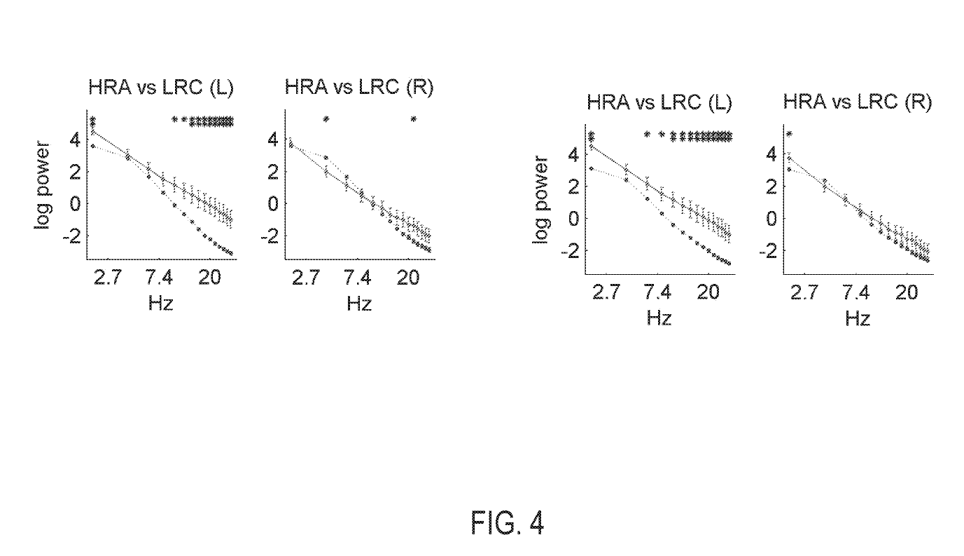

Neonatal EEG showed statistically significant differences in power (log power) across much of the power spectrum (2 to 30 Hz) when comparing HRA infants in sleep (S) to LRC infants in either active sleep (AS) or quiet sleep (QS). This finding was predominantly in the left hemisphere where power (L-PWR) for the HRA group was significantly higher than LRC L-PWR across the majority of the power spectrum [FIG. 4], whereas right hemisphere power (R-PWR) showed significant differences between the two groups for only two frequencies. Since QS generally showed higher power than AS in the LRC group except at frequencies above 15 to 20 Hz, and sleep was not parsed in the HRA group, L-PWR comparisons between HRA-S and LRC-QS is the more stringent test. In this analysis, L-PWR for HRA-S was significantly higher than LRC-QS at very low frequencies 2 Hz (p</=0.01), high frequencies from 14 to 30 Hz (p</=0.01), and midrange frequencies from 10-12 Hz (p</=0.05) revealing a marked hemispheric asymmetry in power.

TABLE 4b
HRA Cohort with 72-month outcome

| | | Age at PDD- |
|---|---|---|
| Subject # | Gender | BI (mo) |
| 10 | F | 70.36 |
| 11 | M | 72.63 |
| | | 71.04 |

TABLE 5a
Outcome for subjects completing protocol assessments at 20 and 30 months

| | | 20 mo | | | 30 mo | | | | |
|---|---|---|---|---|---|---|---|---|---|
| | | ADOS | | | ADOS | | | | |
| | | Total | ADOS | | Total | ADOS | | ADOS CSS | ADOS |
| N | Sex | Score | Classification | CSS | Score | Classification | CSS | delta 20-30 mo | Dx |
| 1 | F | 1 | NS | 1 | 3 | NS | 2 | plus 1 | NS |
| 4 | F | 12 | AS | 4 | 9 | AS | 4 | 0 | AS |
| 3 | F | nd | nd | nd | 3 | NS | 1 | na | NS |
| 5 | M | 6 | NS | 1 | 5 | NS | 2 | plus 1 | NS |
| 6 | M | 6 | NS | 1 | 14*/10 | A*/AS | 7*/4 | plus 3 | AS |
| 7 | M | 5 | NS | 2 | 6 | NS | 2 | 0 | NS |
| 9 | M | 15 | AS | 5 | 21 | A | 9 | plus 4 | A |
| 10 | M | 5 | NS | 1 | 8 | AS | 4 | plus 3 | AS |
| 11 | M | 13 | AS | 4 | 2 | NS | 1 | minus 3 | NS |

*Scores from 24 & 30 month assessments added due to clinical regression.

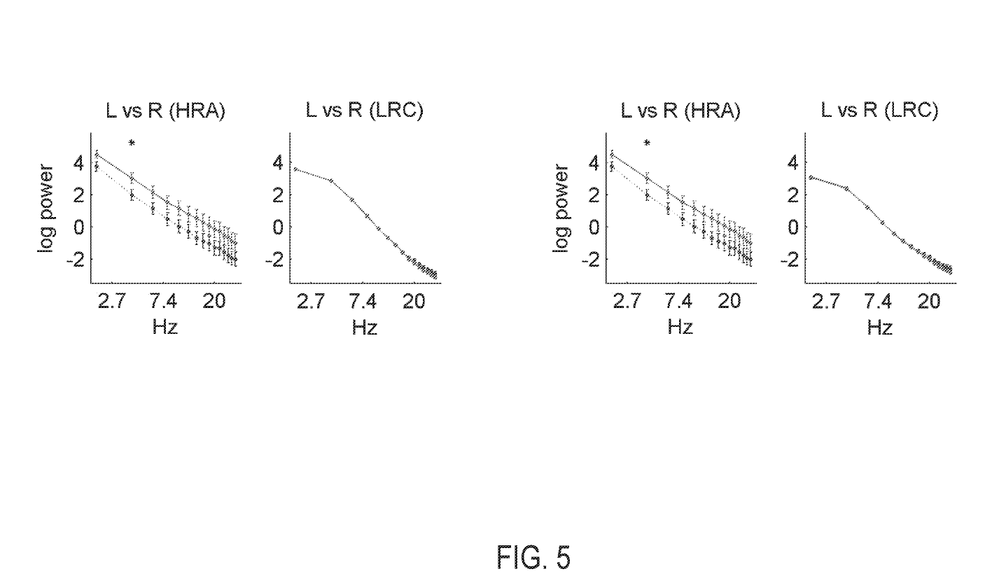

The error bars for the HRA and LRC cohort are shown. LRC error bars are so small as to be difficult to discern. In the RH, log power was generally much more similar between the two groups though some discrete differences reached significance. HRA power in the R-pwr was significantly higher than LRC R-pwr at 22 Hz (p</=0.05) when compared to QS and at 2 Hz when compared to AS. Interestingly, in only one instance was HRA power lower than the LRC cohort and this occurred in the RH at 4 Hz (p</=0.05) for the QS comparison but the finding did not hold up in the AS comparison. The asymmetry of power between the hemispheres is apparent in FIG. 5 where L-R asymmetry is noted to be present across the power spectrum for the HRA group in sleep and reached significance at 4 Hz (p=0.0429). By comparison, the LRC group showed essentially no hemispheric asymmetry in either QS or AS.

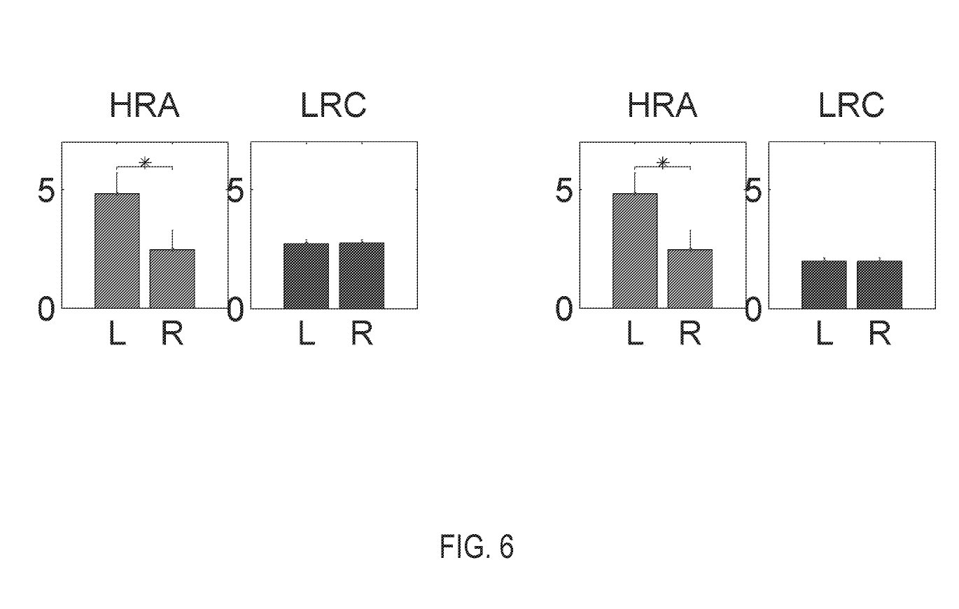

An analysis of L-R asymmetry from 2 to 20 Hz (integrated log power) [FIG. 6], further illustrates the significance of the asymmetry for the HRA group (p</=0.05) over a large part of the power spectrum.

Higher within group variability in power is also noted across the power spectrum in the HRA compared to the LRC group (see FIG. 4 error bars). Indeed, we took the mean of the standard errors over the spectrum in each group and found highly significant differences between groups in both hemispheres (HRA variability >LRC variability, p values <10⁻¹⁰). This finding demonstrates a heterogeneity among the HRA subjects not present in the LRCs. This variability did not undermine the ability to find highly significant differences in power between the two groups.

Neonatal EEG Findings: HRA Vs. LRC Coherence—Group Data

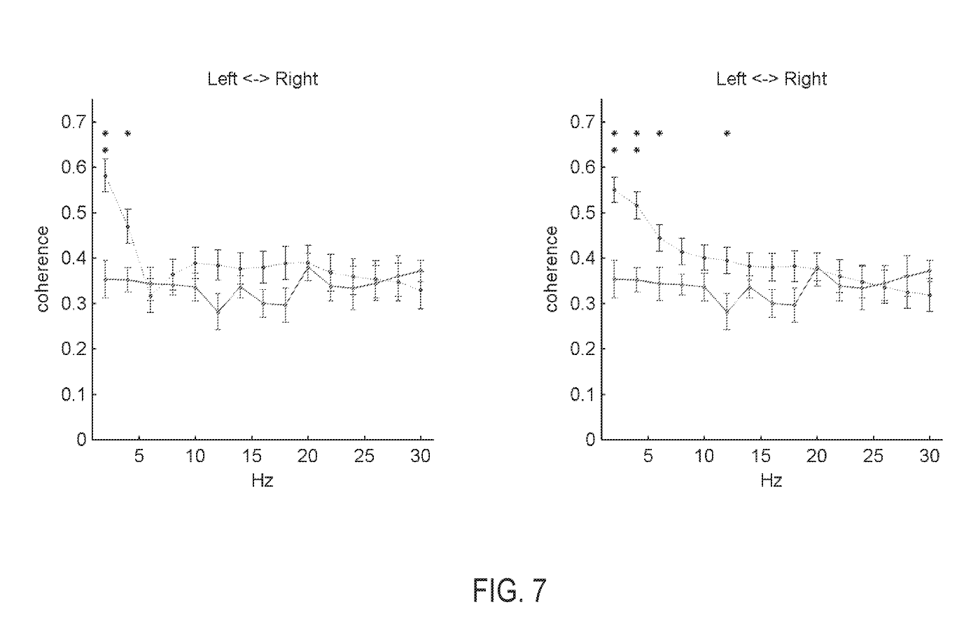

Neonatal EEG showed statistically significant differences in inter-hemispheric coherence L-R/C) when comparing HRA infants to LRC infants in either AS or QS. [FIG. 7] Using QS for comparison, L-R/C was significantly lower in the HRA group compared to the LRC group at 2 Hz (p</=0.01) and 4 Hz (p</=0.05). Using AS for comparison LR/C showed greater difference, significantly lower at 2 and 4 Hz (p</=0.01) and 6 and 12 Hz (p</=0.05). Volume conduction between hemispheres could not be computed given the number of leads used. However a comparison of group data for low frequency waves 2-4 Hz to 16-30 Hz coherence, as a proxy for volume conduction, indicates that inter-hemispheric functional connectivity is largely absent in the HRA group. As illustrated by the LRC cohort in the normal quiet sleeping (QS) condition, the greatest coherence in sleep normally occurs in the low frequencies, i.e. 4 Hz and less. By comparison, the HRA cohort is showing essentially no coherence in the low frequencies, suggesting no functional connectivity in sleep in the HRA group's neonatal EEG. This is supported by intra-subject comparison of slow wave (2-4 Hz) coherence to high frequency [16-30 Hz] coherence where LRCs showed increased coherence in low frequencies compared to their own high frequency coherence but HRA subjects failed to show any significant coherence

TABLE 5b
Outcome for subjects completing PDD-BI

| | | 20 mo | | 30 mo | | 72 mo | | |
|---|---|---|---|---|---|---|---|---|
| | | PDD-BI | AUC | PDD-BI | AUC | PDD-BI | AUC | PDD-BI |
| | | AUC | Dx | AUC | Dx | AUC | Dx | AUC clas- |
| N | Sex | T-score | Class | T-score | Class | T-score | Class | sification |
| 1 | F | 10 | NS | 18 | NS | ∘ | ∘ | NS |
| 2 | F | 76 | A | 77 | A | ∘ | ∘ | A |
| 3 | F | nd | nd | ∘ | ∘ | 32 | PDD | PDD |
| 4 | F | nd | ∘ | 36 | PDD- | ∘ | ∘ | PDD- |
| | | | | | NOS | | | NOS |
| 5 | M | 81 | A | 70 | A | ∘ | ∘ | A |
| 6 | M | 20 | NS | 44 | A | ∘ | ∘ | A |
| 7 | M | 17 | NS | 18 | NS | ∘ | ∘ | NS |
| 8 | M | nd | nd | ∘ | ∘ | 19 | NS | NS |
| 9 | M | 40 | PDD- | 49 | A | ∘ | ∘ | A |
| | | | NOS | | | | | |
| 10 | M | 31 | NS | 35 | PDD- | ∘ | ∘ | PDD- |
| | | | | | NOS | | | NOS |
| 11 | M | 22 | NS | 38 | PDD- | ∘ | ∘ | PDD- |
| | | | | | NOS | | | NOS |

### II. Developmental Outcome of HRA Cohort

All eleven infants in the HRA cohort had diagnostic outcome assessments. Nine completed the study protocol with full outcome measures at 30 months of age (Mean age at assessment: 30.65 mo) [Table 5a.] Eight of those nine also had assessments at 20 months (Mean age at assessment: 21.29 (N=8). The two subjects without 30-month outcome assessments later completing a limited diagnostic outcome assessment at 72 months of age (mean age at assessment: 71.04 months.) [Table 5b.] The outcome results for ADOS, PDD-BI Autism Composite (AUC) and Social Discrepancy (SOCDSC) are presented in Tables 6a, b and c. A “Best Estimate Diagnostic Classification” was determined using ADOS outcome at 30 months, informed by PDD-BI AUC and SOCDSC scores at 30 mo. [Table 7.] In the case of disagreement between the instruments, ADOS diagnostic classification was deemed most valid with evidence from PDD-BI often adding information that suggested a diagnosis of broader autism phenotype, designated NS-BAP.

TABLE 5c
Outcome for subjects completing PDD-BI

| | | 20 mo | | 30 mo | | 72 mo | | PDD-BI |
|---|---|---|---|---|---|---|---|---|
| | | PDD-BI | SOCDSC | PDD-BI | SOCDSC | PDD-BI | SOCDSC | SOCDSC |
| | | SOCDSC | Dx Cl | SOCDSC | Dx Cl | SOCDSC | Dx Cl | DX |
| N | Sex | 20 mo. | @ 20 mo | @ 30 mo. | 30 mo | 72 mo | @ 72 mo | Classification |
| 1 | F | 67 | NS | 39 | NS | ∘ | nd | NS |
| 2 | F | 53 | NS | 35 | NS | ∘ | nd | NS |
| 3 | F | ∘ | ∘ | ∘ | ∘ | −41 | NS | NS |
| 4 | F | ∘ | ∘ | 23 | ASD | ∘ | nd | ASD |
| 5 | M | 60 | NS | 22 | ASD | ∘ | nd | ASD |
| 6 | M | 68 | NS | 20 | ASD | ∘ | nd | ASD |
| 7 | M | 55 | NS | 44 | NS | ∘ | nd | NS |
| 8 | M | ∘ | ∘ | ∘ | ∘ | 43 | NS | NS |
| 9 | M | 15 | ASD | 6 | ASD | ∘ | nd | ASD |
| 10 | M | 43 | NS | 41 | NS | ∘ | nd | NS |
| 11 | M | 29 | NS | 13 | ASD | ∘ | nd | ASD |

Of the eleven infant siblings to complete the study, two developed Autism and two more met criteria for an Autism Spectrum disorder (36%). Both subjects diagnosed with Autism were male and both had regressions documented based on change in diagnosis on the ADOS between 20 and 30 months of age. One had a rapid regression between 20 and 24 month precipitating an additional assessment at 24 months of age (Subject 6). One of the other two subjects diagnosed with an ASD, a male, also had a regression between 20 and 30 months of age. The other subject with ASD, a female, met criteria on ADOS at 20 months and did not show a regression. Thus the risk of developing autism in the HRA males was greater than the risk to HRA females and no female had a regression or developed full Autistic Disorder.

TABLE 6
Best Estimate diagnostic classification based on
ADOS and PDD-BI AUC and SOCDSC

| | | | PDD-BI | PDD-BI | |
|---|---|---|---|---|---|
| | | ADOS | AUC | SOCDSC | Best |
| | | Classification | Classification | Classification | Estimate |
| Subject | Sex | @ 30 mo | @ 30 mo | @ 30 mo | Dx |
| 1 | F | NS | NS | NS | NS |
| 2 | F | AS | AS | NS | ASD |
| 3 | F | nd | PDD-NOS | NS | NS/BAP |
| 4 | F | NS | PDD-NOS | ASD | NS/BAP |
| 5 | M | NS | A | ASD | NS/BAP |
| 6 | M | A/AS# | A | ASD | A |
| 7 | M | NS | NS | NS | NS |
| 8 | M | nd | NS## | NS## | NS |
| 9 | M | A/ASD# | A | ASD | A |
| 10 | M | AS | PDD-NOS | NS | ASD |
| 11 | M | NS | PDD-NOS | ASD | NS/BAP |

Of the seven subjects that did not meet criteria for an ASD, three with a NS ADOS diagnosis at 30 months had parental concerns at the same time about behavior as measured on the PDD-BI and were judged Non-Spectrum/Broader Autism Phenotype (NS-BAP). A fourth subject (subject #4) with no ADOS score had clinical concerns at 72 months regarding language development but no social concerns and the PDD-BI score for AUC was at the cut off for PDD-NOS, while SOCDSC was not indicative of an ASD. Best estimate was that she too fit criteria for NS-BAP. In the other three subjects, ADOS classification and PDD-BI were in agreement as NS in two, and the third assessed at 72 months of age as NS had no clinical concerns on history and no concerns revealed by the PDD-BI.

TABLE 7
Comparison of diagnostic for each HRA infant
sibling and their older sibling/Proband

| Subject | Subject | | | Proband |
|---|---|---|---|---|
| # | Gender | HRA BstEstDx | Proband Gender | BstEstDx |
| 1 | F | NS | M | ASD |
| 2 | F | ASD | M | Autism/ASD |
| 3 | F | NS/BAP | M | Autism |
| 4 | F | NS/BAP | M | Autism |
| 5 | M | NS/BAP | M | Autism/ASD |
| 6 | M | A | F | Autism |
| 7 | M | NS | M | ASD |
| 8 | M | NS | M | ASD |
| 9 | M | A | M | Autism |
| 10 | M | ASD | M | Autism |
| 11 | M | NS/BAP | M | Autism |

Nine of the 11 HRA cohort were assessed for delays in development with the MSEL at 30 months [Table 9]. Verbal and Non-verbal DQs varied widely. As a group, visual recognition was the strongest domain and fine and gross motor domains were the weakest. Non-verbal DQ was impacted by the low fine motor scores. One subject (Subject 2) stood out due to significant delays across all domains with a Verbal DQ of 55.33, well below her Non-verbal DQ of 74.55. This subject also met criteria for ASD at 20 and 30 months by ADOS. She, like two of the other three subjects with ASD and two of the three subjects with NS-BAP, showed lower verbal scores compared to Visual Recognition. The forth subject with an ASD had the reverse profile suggestive of a nonverbal learning disability (NLD). One additional subject not on the spectrum also showed stronger verbal than non-verbal performance suggestive of an NLD profile.

Eight of the 11 HRA cohort were assessed at 30-month outcome for adaptive skill level using the Vineland Adaptive Behavior Scales (VABS) [Table 9]. Adaptive skills Composite (ABC) for the group ranged from 70 (Borderline Adaptive Functioning to 114 (High Average Adaptive Functioning) with a mean in the low average range. As a group, the highest adaptive skills domain was for Motor skills with the lowest adaptive domain in Socialization.

TABLE 8
Mullen Scales of Early Learning standard scores for
HRA cohort at 30-month outcome with ASD BstEstDx

| | | | | | | | Non- | |
|---|---|---|---|---|---|---|---|---|
| Subject | Rec | Exp | Vis | Fine | Gross | Verbal | verbal | |
| # | Lang | Lang | Rec | M | M | DQ | DQ | BstEstDx |
| 1 | 111.19 | 131.4 | 138.14 | 114.56 | 90.97 | 121.29 | 126.35 | NS |
| 2 | 62.96 | 49.7 | 82.84 | 66.27 | 66.27 | 56.33 | 74.55 | ASD |
| 3 | — | — | — | — | — | — | — | NS/BAP |
| 4 | 88.94 | 83.01 | 133.41 | 80.05 | 68.19 | 85.98 | 106.73 | NS/BAP |
| 5 | 92.78 | 72.9 | 109.34 | 89.46 | 76.21 | 82.84 | 99.4 | NS/BAP |
| 6 | 111.04 | 117.77 | 121.13 | 90.85 | 94.21 | 114.4 | 105.99 | A |
| 7 | 119.05 | 122.35 | 109.13 | 99.21 | 92.59 | 120.7 | 104.17 | NS |
| 8 | — | — | — | — | — | — | — | NS |
| 9 | 91.38 | 55.48 | 110.97 | 71.8 | 81.59 | 73.43 | 91.38 | A |
| 10 | 107.79 | 125.17 | 90.4 | 79.97 | 111.27 | 116.48 | 85.19 | ASD |
| 11 | 90.27 | 80.24 | 90.27 | 80.24 | 76.9 | 85.26 | 85.26 | NS/BAP |
| Means | 97.27 | 93.11 | 109.51 | 85.82 | 84.24 | 95.19 | 97.67 | |

III. Correlation Analyses: HRA Neonatal EEG Measures: L-R Hemisphere Coherence and L & R Hemisphere Power, Vs. Developmental Outcome Measures

Color-coded matrices (“heat maps”) are used here to display the results of correlation analyses between EEG signature in the neonatal period and behavioral outcome measures at 30+ months. For all heat maps, the x-axis is frequencies 2-30 Hz in 2 Hz intervals. The y-axis is categorical for outcome assessment instrument domains and composites. For each correlation there are two heat maps: correlation coefficient (CC) on the left and P values, represented here as [1 minus P] on the right. Per the legend for correlation coefficient, a positive correlation falls in the “red” spectrum, and a negative correlation falls in the “blue” spectrum. Per the legend for P values, the most significant P values fall in the “red” spectrum with green representing the p=0.05 cut-off (1−p=0.95).

TABLE 10
Vineland Adaptive Behavior standard scores for
HRA cohort at 30-month outcome with BstEstDx

| Subject # | Gender | Comm-C | DLS-C | SOC-C | Motor-C | ABC | BestEstDx |
|---|---|---|---|---|---|---|---|
| 1 | F | 110 | 107 | 111 | 117 | 114 | NS |
| 2 | F | 71 | 78 | 68 | 77 | 70 | ASD |
| 3 | F | — | — | — | — | — | NS/BAP |
| 4 | F | — | — | — | — | — | NS/BAP |
| 5 | M | 76 | 78 | 74 | 79 | 73 | NS/BAP |
| 6 | M | 110 | 100 | 96 | 105 | 103 | A |
| 7 | M | 101 | 98 | 100 | 96 | 98 | NS |
| 8 | M | — | — | — | — | — | NS |
| 9 | M | 76 | 75 | 72 | 90 | 75 | A |
| 10 | M | 101 | 104 | 100 | 111 | 105 | ASD |
| 11 | M | 97 | 100 | 93 | 102 | 97 | NS/BAP |
| Mean 91.8 | | 92.8 | | 92.5 | 89.3 | 97.1 | |

HRA Correlations: EEG Power and Coherence Vs. Best Estimate Diagnosis (BstEstDx) (N=11)

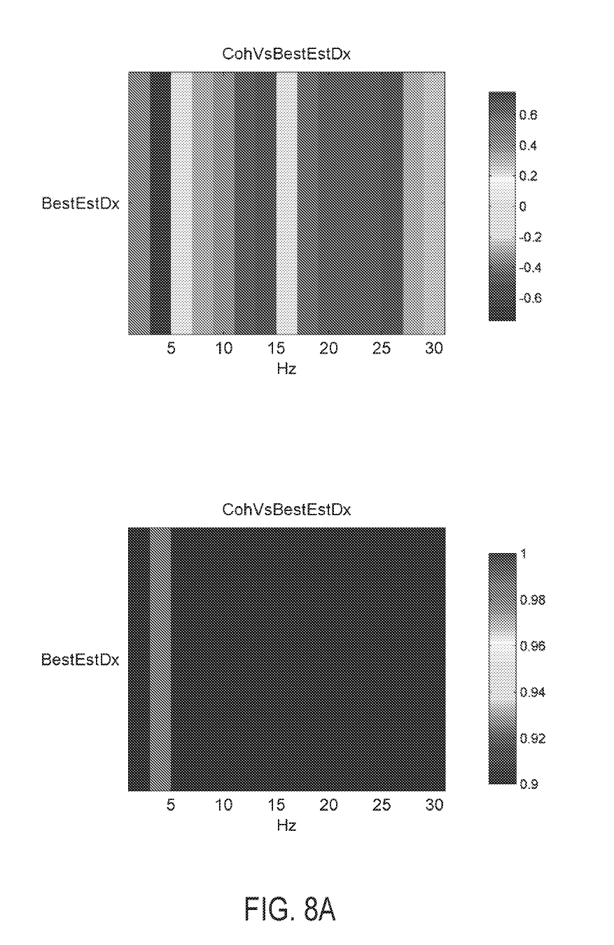

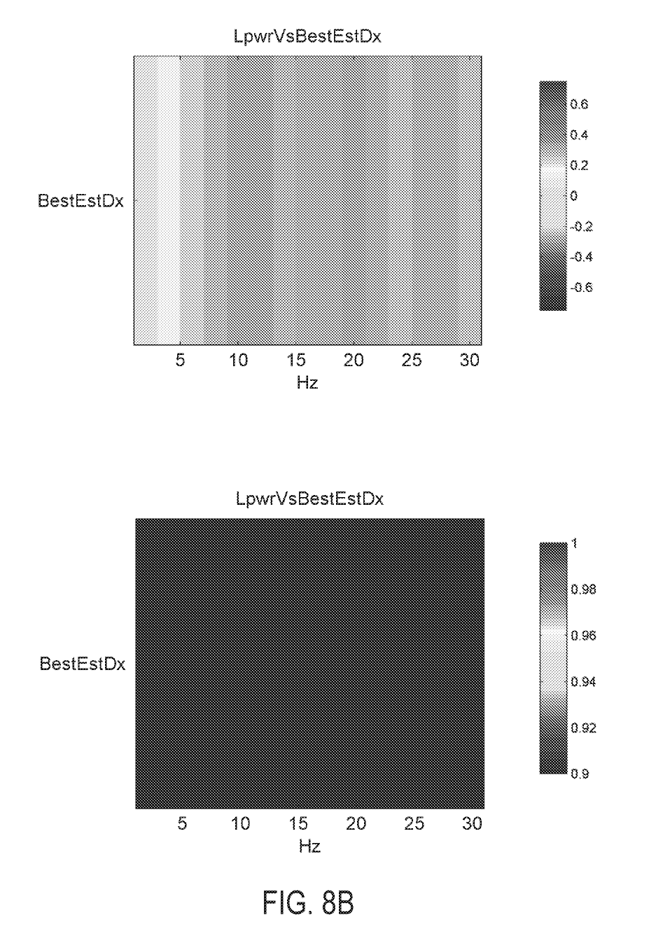

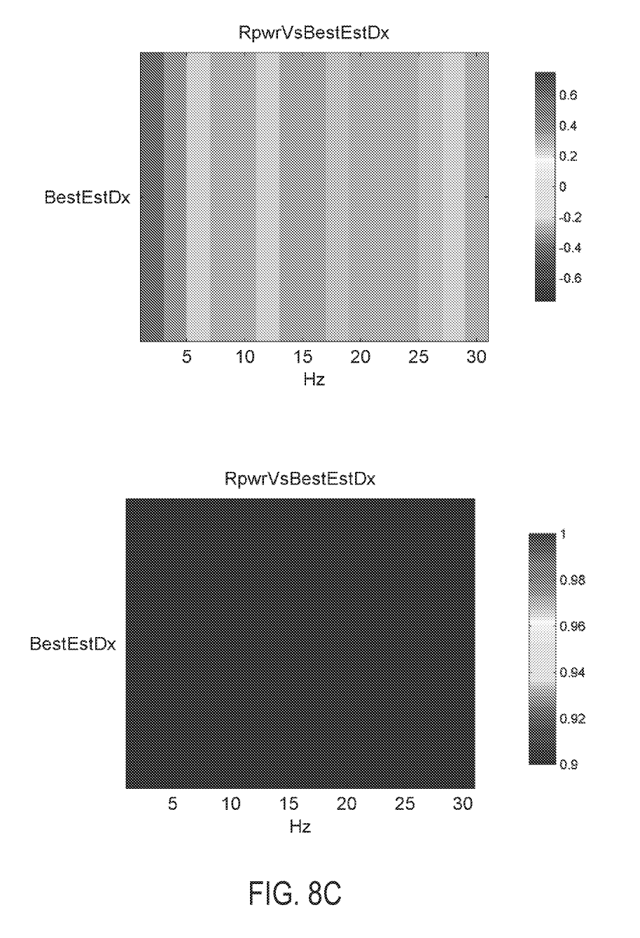

Correlation analysis for neonatal EEG L-R coherence vs. BstEstDX using a graded severity rating for each classification: Autism (4), ASD (3), NS-BAP (2) and Non-Spectrum (1) revealed significant negative correlation which reached high significance at 4 Hz (CC: 0.717, P=0.013), demonstrating that in this sample, the severity of an ASD classification at 30 months of age was predicted by the degree to which coherence was decreased in the sleeping infant soon after birth [FIG. 8A]. No significant correlations with BstEstDx were found for neonatal spectral power in Left or Right Hemispheres, but it is important to note that L-pwr shows generally positive correlation with BstEstDx, while R-pwr is showing a negative correlation.

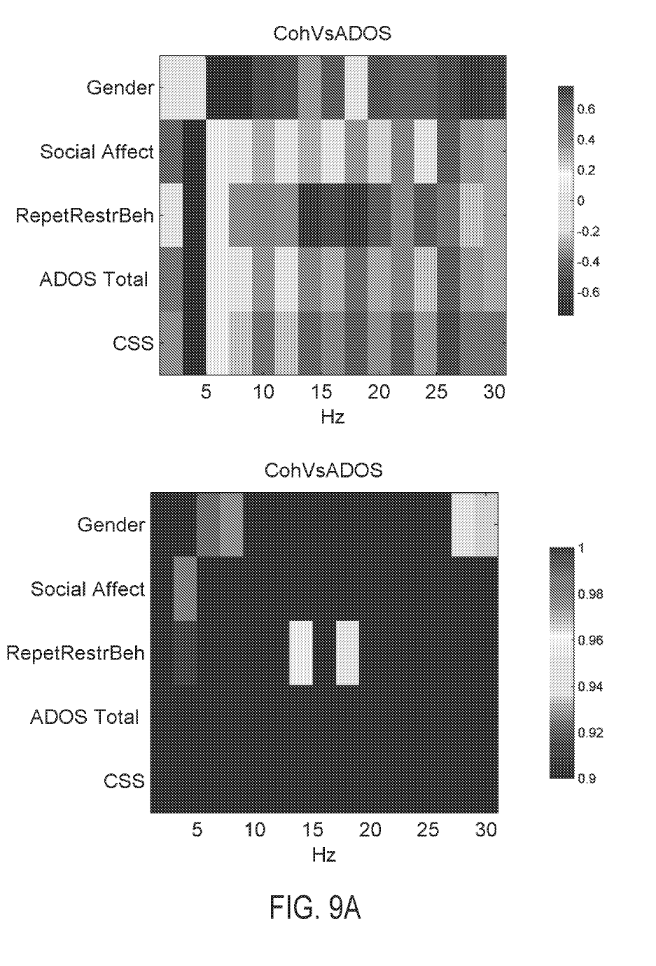

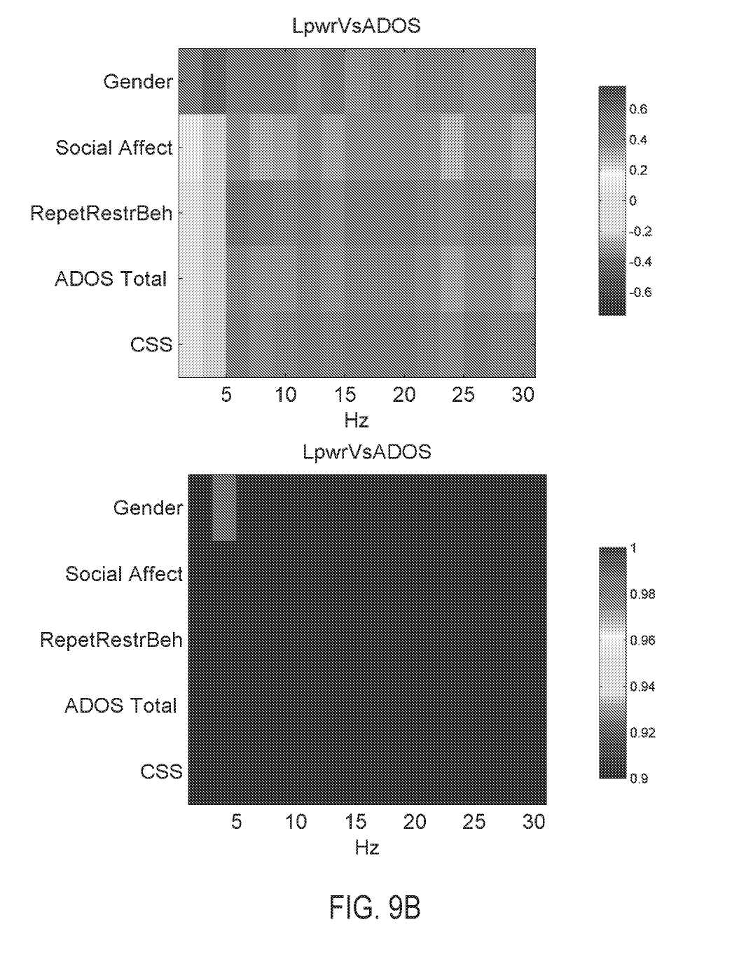

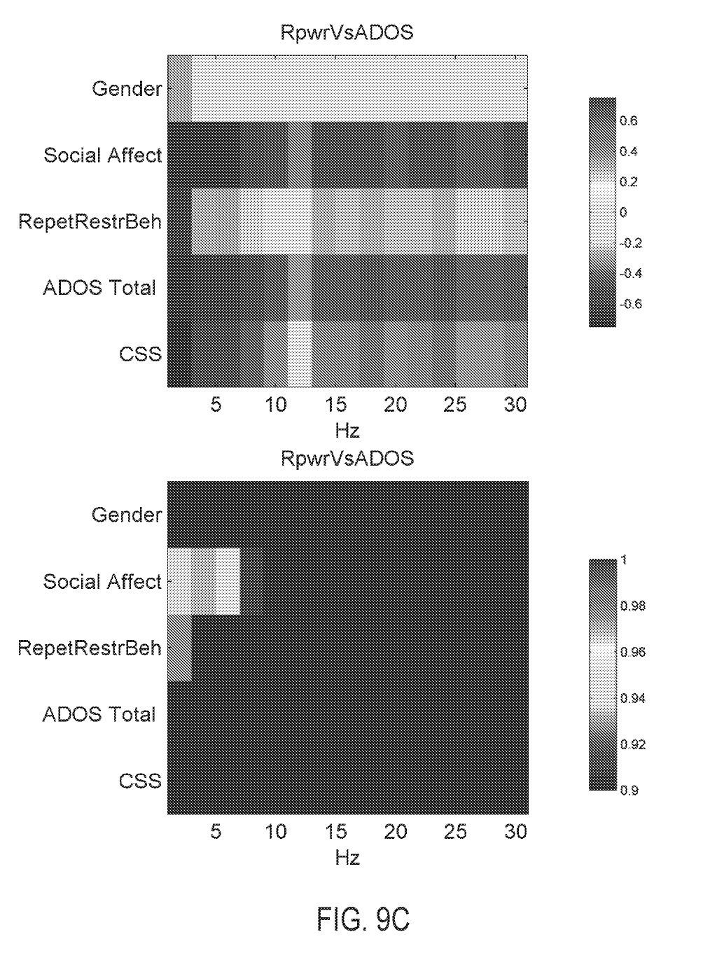

Correlation analysis of the neonatal Coherence as a predictor of ADOS outcome at 30-month revealed a negative correlation for Social Affect (SA) and Repetitive-Restrictive Behaviors (RRB) subdomains, the Total ADOS score and the ADOS severity score (CSS). [FIG. 9A.] RRB score was profoundly negatively correlated with Coherence at 4 Hz (CC: −0.92, p=0.0005) as well as at 14 and 16 Hz. SA score was also highly negatively correlated with Coherence at 4 Hz (CC: −0.78, p=0.014). ADOS Total score and CSS though highly negatively correlated, failed to reach statistical significance. These results again point to decreased Coherence in the newborn period as a predictor of later ASD behavior, with decreasing Coherence correlated with increasing severity. L-pwr showed a positive though statistically non-significant correlation with the ADOS [FIG. 9B]; however, R-pwr [FIG. 9C] showed a strong negative correlation with SA, which reached significance at 4 Hz (CC: −0.72, p=0.031), 2 Hz CC: −0.70, p=0.035) and 6 Hz (CC: −0.67, p=0.047). Since SA on the ADOS increases with increased severity of ASD, these findings indicate that herein lower R-pwr in the neonatal period was associated with worse social affective behaviors and an increased likelihood of ASD at 30-month outcome.

The PDD-BI has fifteen domain and composite scores that are divided into three categories: 1) POSITIVE SYMPTOMS: seven domain scores and their two derived composites that increase as autism severity increases, and 2) NEGATIVE SYMPTOMS: three domain scores and their two derived composites that decrease as autism severity increases, 3) the composite for Autism diagnosis/severity, calculated by algorithm including the most critical positive and negative symptoms, which yields a score that increases as autism severity increases, and 4) the Social Discrepancy Score computed from negative and positive social domain scores which decreases as autism severity increases.

### Coherence and PDDBI Correlations:

PDD-BI Positive Symptoms and their Composites:

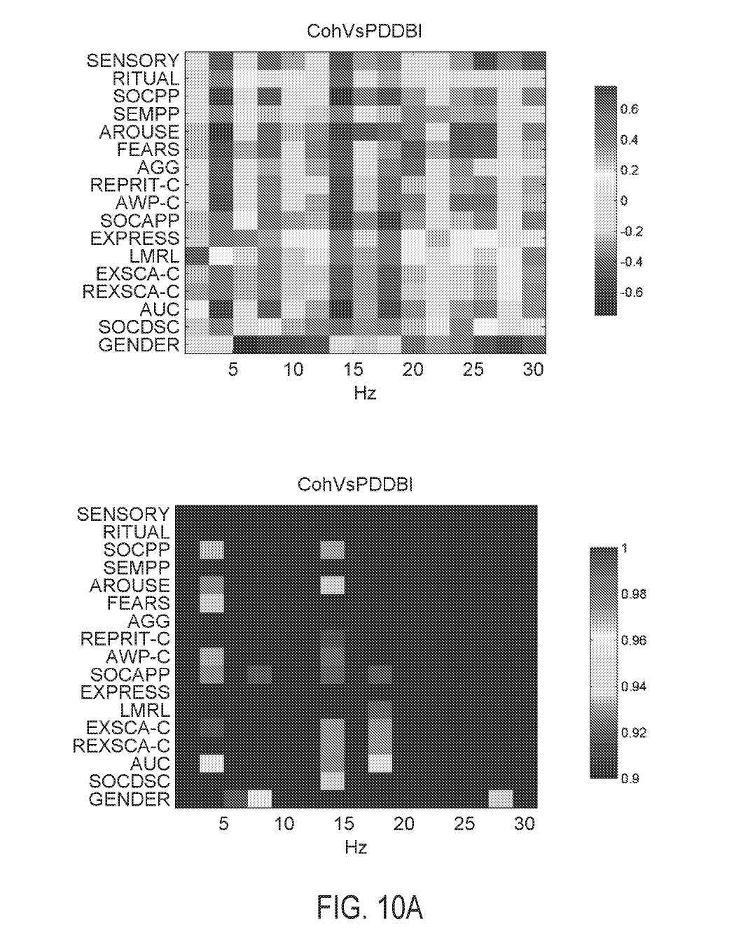

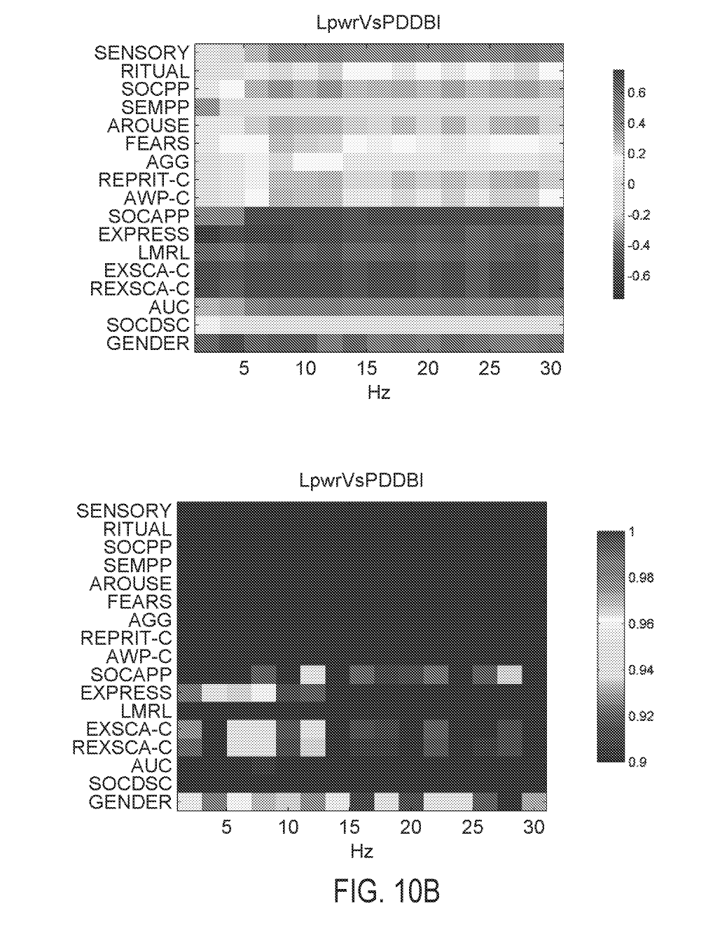

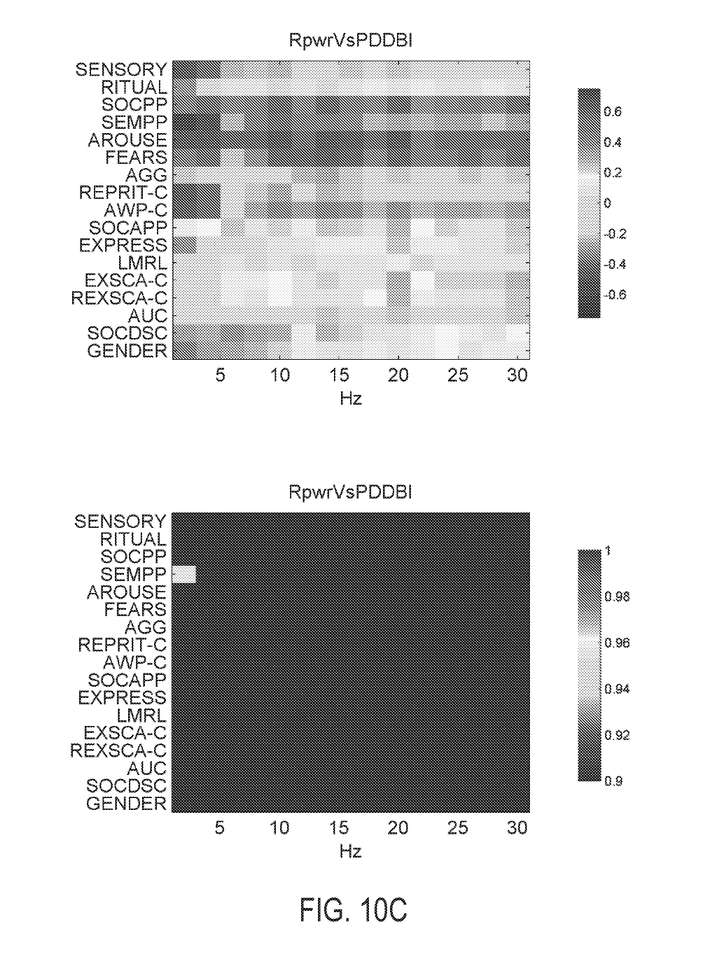

PDD-BI positive symptoms were negatively correlated with neonatal coherence [FIG. 10A—rows 1-9], with the significant negative correlations at 4 Hz and 14 Hz for both Social Pragmatic Problem (SocPP) (4 Hz: CC: −0.65, p=0.032, 14 Hz: CC: −0.66, p=0.027) and Arousal regulation (AROUSE) (4 Hz: CC: −0.68, p=0.022, 14 Hz: CC: −0.64, p=0.034). Positive symptoms for sensory, specific fears, aggressiveness and Semantic/Pragmatic problems though generally negatively correlated, did not show significant correlations.

PDD-BI Negative Symptoms and their Composites:

PDD-BI negative symptoms were positively correlated with neonatal coherence from 4-30 Hz, but negatively correlated at 2 Hz [FIG. 10A—rows 10-14]. Strongest positive correlation was seen at 14 and 16 Hz for Social Approach (14 Hz: CC: 0.77, p=0.005, 16 Hz: CC: 0.78, p=0.005) which was the principle contributor to the significance of the positive correlations seen for the negative symptom composites EXSCA-C (14 Hz: CC 0.67, p=0.025, 16 Hz CC: 0.67 p=0.025) and REXSCA-C (14 Hz CC: 0.66 p=0.026; 16 Hz CC: 0.66 p=0.026) Expressive Language (EXPRESS) and Learning, Memory and Receptive language (LMRL) did show positive correlations, especially at 14 and 16 Hz which contributed to these composites, but both failed to reach significance independently at any frequency.

### PDD-BI Autism Composite:

PDD-BI Autism Composite was negatively correlated with neonatal coherence [FIG. 10A—row 15] from 4 to 30 Hz reaching significance at 4 and 14 Hz (4 Hz: CC −0.62, p=0.043, 14 Hz CC: −0.67, p=0.023).

### PDD-BI Social Discrepancy:

PDD-BI Social Discrepancy was poorly correlated with neonatal coherence [FIG. 10A—row 16], failing to achieve significance at any frequency. Again, as evidenced by both PDD-BI positive and negative symptoms and the Autism Composite, decreased EEG coherence in the neonate was predictive of worse autistic symptoms at outcome, in this case as related to Social Pragmatics, Arousal Regulation and Social Approach behaviors, to Autistic Behaviors, broadly, as measured by the Autism Composite and, to a lesser extent, language development.

### LH-Pwr and PDD-BI Correlations:

PDD-BI Positive Symptoms and their Composites:

PDD-BI positive symptoms and their composites were generally positively correlated with L-pwr, especially SENSORY, but failed to reach significance at any frequency.

PDD-BI Negative Symptoms and their Composites:

PDD-BI negative symptoms and their composites were generally negatively correlated (FIG. 10B) with coherence and reached high significance for Expressive language (EXPRESS) at 2 Hz (CC: −0.72, p=0.011) and at 6 Hz and 8 Hz (CC: −0.64, p=0.034, CC: −0.62, p=0.042, respectively). The EXSCA-C also achieved significance at the 0.05 level at 6 Hz, and bordered on significance at 8 Hz. Similarly, the REXSCA-C composite bordered on significance at 6 and 8 Hz.

### PDD-BI Autism Composite and Social Discrepancy:

Neither the Autism composite nor Social Discrepancy was significantly correlated with Lpwr, although AUC did show a positive correlation.

### RH-Pwr and PDD-BI Correlations:

### PDD-BI Symptoms and Composites

PDD-BI symptoms and composites [FIG. 10C.] showed weakly positive correlation with negative symptoms and weakly negative correlation with positive symptoms, but no significant correlations for any symptom at any frequency. As evidenced here by the PDD-BI negative symptoms, increased EEG power in the L

HRA Correlations: EEG Power and Coherence Vs. Mullen Scales of Early Learning (MSEL) (N=9)

The MSEL has five developmental domains: Receptive and Expressive Language, Fine and Gross motor and Visual Recognition, and 2 Developmental Quotients (DQ): Verbal and Non-verbal.

### Coherence and MSEL Correlations:

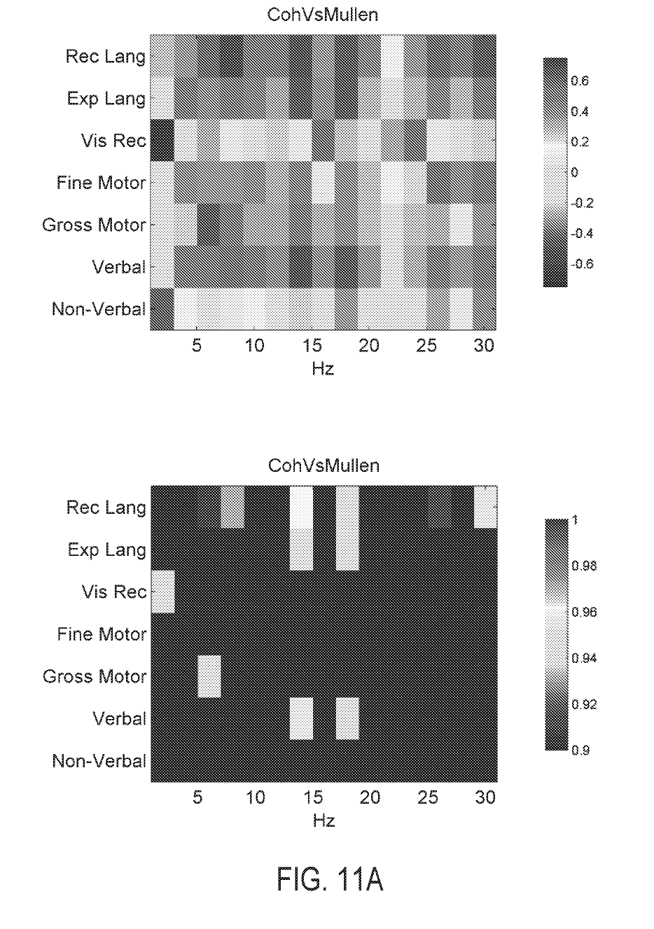

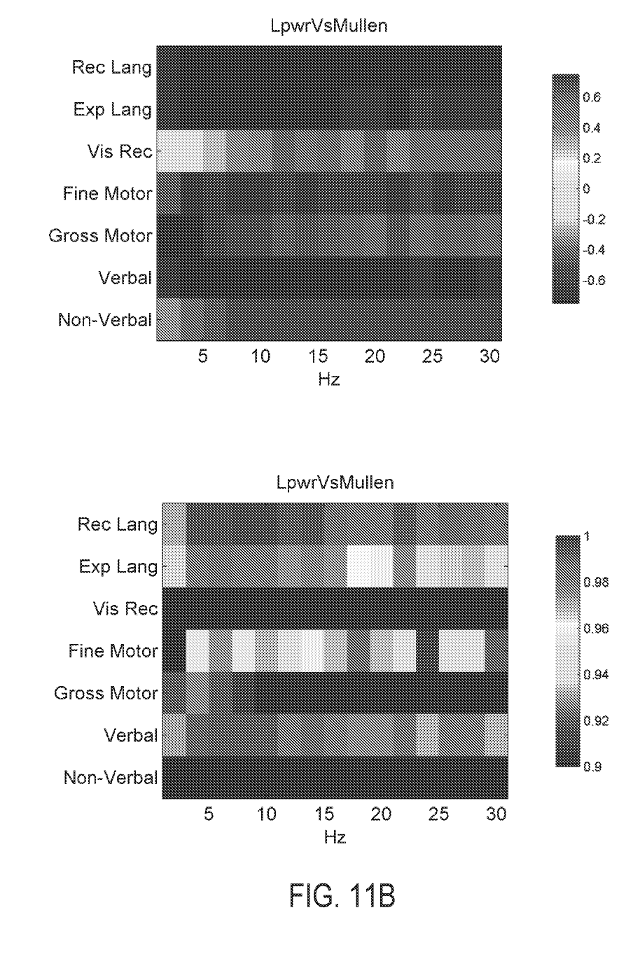

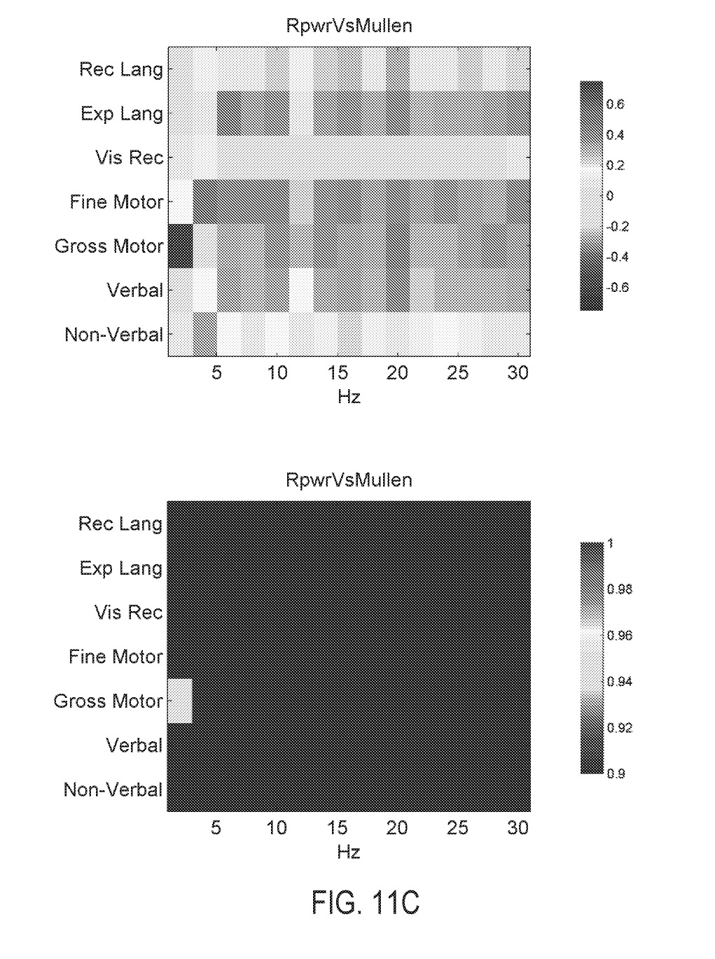

MSEL scores showed positive correlations with neonatal coherence for the language domains, the Verbal DQ and for the Motor domains [FIG. 11A]. Receptive language (RL) showed the most significant correlations with high CC at 8, 14, 16 and 30 Hz; all but one achieving statistical significance. The highest significance was at 8 Hz (CC: 0.72, p=0.029), followed by 14 Hz (CC: 0.69, p=0.038) and 30 Hz (CC: 0.67, p=0.047). Expressive language (EL) was also significantly correlated at one frequency, 14 Hz (CC: 0.67, p=0.048). These combined to yield a significant result for the VDQ at 14 Hz (CC: 0.70, p=0.037) and borderline significance at 16 Hz (CC: 0.66, p=0.051). The only Motor domain achieving significance was Gross Motor and only at 6 Hz (CC: 0.67, p=0.049). NO significance was found for any frequency for Fine Motor or for Non-verbal DQ. Interestingly, Visual recognition showed a negative correlation at 2 Hz, but it failed to attain significance.

### LH-Pwr and MSEL Correlations:

The most dramatic findings for the MSEL were in the Left Hemisphere power correlations where highly negative correlations were found for both Language domains and the Verbal DQ and to a lesser extent Motor domains [FIG. 11B]. LH-Power showed profoundly significant negative correlations with MSEL RL and EL standard scores across the entire power spectrum studied, i.e. 2 and 30 Hz, short of two frequencies for EL: 22 and 30 Hz. The top three correlations for RL were at 8 Hz (CC: −0.88, p=0.0016), 10 Hz (CC: −0.88, p=0.002) and 4 Hz (CC: −0.85, p=0.004), and the lowest was at 2 Hz (CC: −0.72, p=0.028). For EL the top three correlation were at 6 Hz (CC: −0.79, p=0.011), 8 Hz (CC: −0.78, p=0.013) and 4 Hz (CC: −0.76, p=0.018), with significant CC and p-values for the remaining ten frequencies. L-pwr and Verbal DQ were similar to those found for RL—and were consistently significant across all frequencies.

LH-pwr showed significant negative correlations for Fine and Gross Motor. Fine Motor was significant at 4, 8, 10 and 14 Hz, maximally at 10 Hz (CC: −0.71, p=0.031). Gross Motor was significant at 2 and 4 Hz, maximally at 2 Hz (CC: −0.82, p=0.007). There were no significant findings for L-pwr and Visual recognition or Non-verbal DQ.

### RH-Pwr and MSEL Correlations:

MSEL scores showed generally positive correlation with neonatal R-Pwr [FIG. 11C], but no domain showed significance at any frequency.

HRA Correlations: EEG Vs. Vineland Adaptive Behavior Scales (VABS) (N=8)

The VABS has four developmental domains: Communication (includes RL and EL), Daily Living Skills, Socialization, Motor (Gross and Fine) and an Adaptive Behavior Composite (ABC).

### Coherence and VABS Correlations:

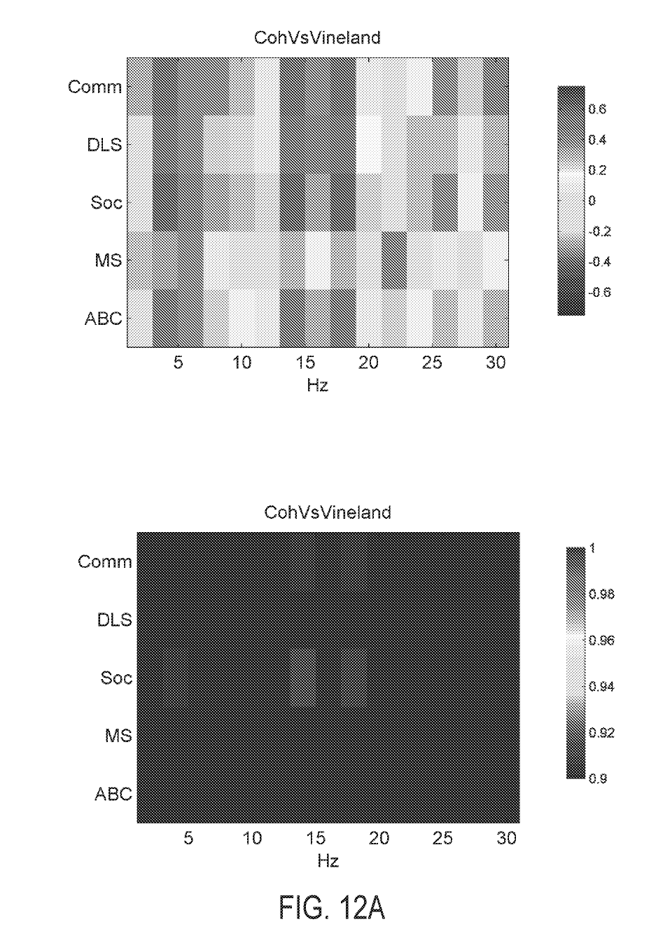

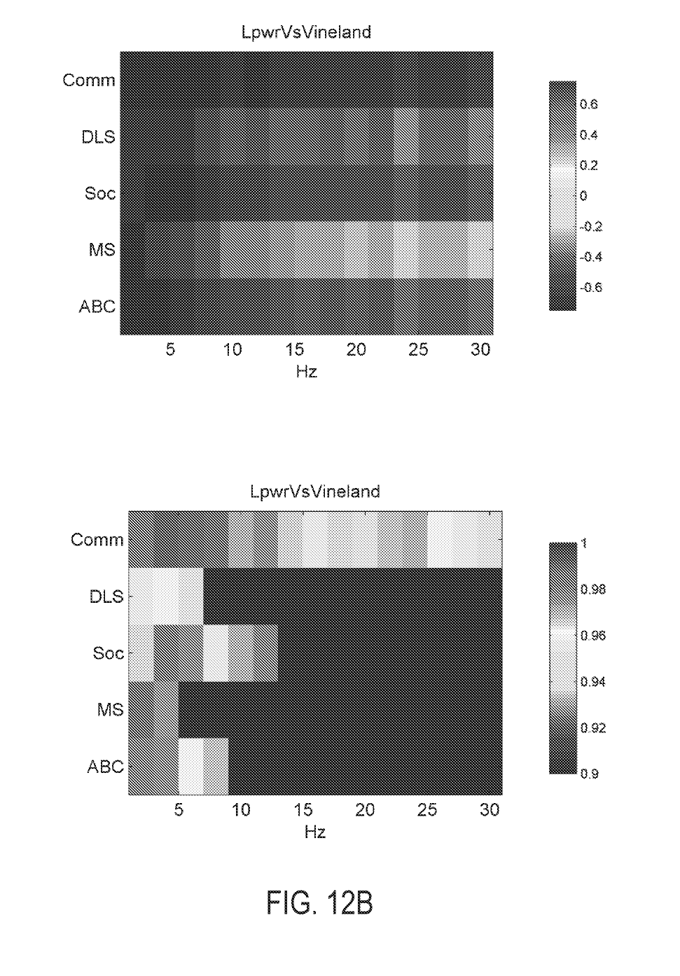

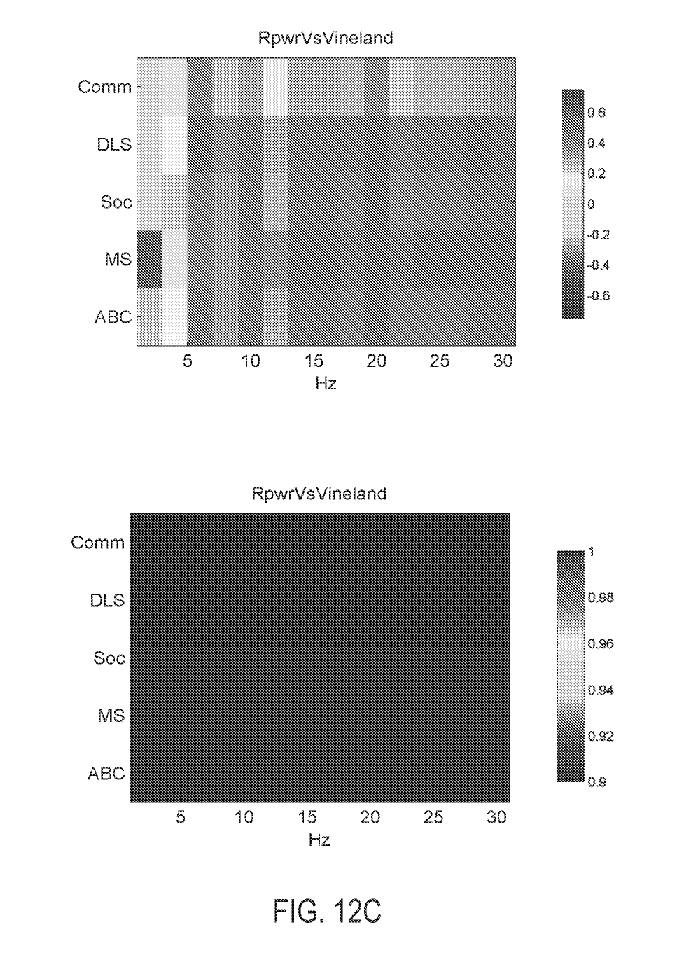

VABS scores showed generally positive correlation with neonatal coherence from 4 to 30 Hz, in particular at 4, 14 and 18 Hz [FIG. 12A], but no domain nor the composite showed significance at any frequency.

### LH-Pwr and VABS Correlations:

VABS scores were negatively correlated with neonatal L-pwr from 2 to 30 Hz [FIG. 12B], with high significance in the Communication (i.e. RL & EL) domain for the majority of the power spectrum, but also in the low frequencies for DLS, SOC, Motor domains and the ABC. The top three correlations for Communication were at 4 Hz (CC: −0.81, p=0.008), 6 Hz (CC: −0.80, p=0.09) and 8 Hz (CC: −0.79, p=0.011), followed closely by significance at 2 Hz, and with no significance at 20, 24 and 30 Hz. DLS, SOC, and ABC all showed significance at 2, 4, and 6 Hz with SOC also significant at 8 Hz. Motor showed significance at 2 Hz only (CC: −0.79, p=0.020). DLS and SOC were most significant at 4 Hz (CC: −0.69, p=0.041)(CC: −0.77, p=0.015, respectively); while the ABC was most significant at 2 Hz (CC: −0.75, p=0.020).

### RH-Pwr and VABS Correlations:

VABS scores showed generally positive correlation with neonatal coherence from 4 to 30 Hz [FIG. 12C], but no domain or DQ showed significance at any frequency. The VABS and the MSEL both showed negative correlations with L power and positive correlations with R Power. And like the MSEL and PDD-BI, the VABS showed strong negative correlations between LH power in the neonatal period and language outcome at 30-month. The negative correlation for SOC vs. L-power at low frequencies on the VABS is in concert with the elevations in L-pwr at 2 Hz. seen in the HRA as compared to the LRA group at 2 wks, but stands in contrast to the lack of correlation between L-pwr and Best Est Dx, ADOS and PDD-BI social behaviors.

### DISCUSSION

Neonatal EEG—Left Power and Inter-Hemispheric Coherence Independently Distinguish HRA Infants from LR Controls in Active and Quiet Sleep.

In this study, sleep EEG power and coherence in the neonatal period (average PMA at EEG: 42.36 weeks) was shown to discriminate healthy term infants at high-risk of developing an ASD (infant siblings of children with ASD) from age-matched, healthy term infants at low risk of developing an ASD on three separate measures. The best discriminator between the two groups was Left Hemisphere Power (uV), which was increased in the HRA infants compared to the LRC across the majority of the power spectrum (QS: p<0.01 at 2, 14-30 Hz, p≤0.5 10-12 Hz) [FIG. 4]. Coherence at low frequencies, delta and theta, was also significantly diminished compared to the control group discriminating between the two groups in slow wave frequencies (2 Hz p<0.01, 4 Hz, p<0.5, respectively) [FIG. 7]. The HRA infants further showed the unexpected finding of marked asymmetry in hemispheric power, significantly higher on the left than on the right hemisphere for the central and occipital regions studied. Low risk controls showed no L-R asymmetry [FIG. 5]. L and R integrated power difference from 2-20 Hz substantiated the finding.

Three parameters distinguished HRA infants from LRC infants: L-power which was elevated for much of the power spectrum, Coherence which was diminished in low frequencies, and L>R power asymmetry which is present in HRA and not in controls. To the best knowledge of the inventors, these are the first biomarkers of any kind shown to discriminate infants at high-risk of ASD from low risk controls in the neonatal period.

Findings for Coherence in the HRA neonates were based on a comparison to LRC in QS and AS and significance was found for both comparisons. Polysomnograms were not conducted to parse AS from QS. While it has been suggested that sleep state needs to be determined to properly measure coherence in the neonate, the statistical significance in either sleep state provides evidence that polysomnograms and sleep parsing into QS and AS are not required to find a significant difference in coherence in the present small cohort. Infants in this study were most likely in AS in that they were recorded immediately after falling asleep, a period when AS is most common in the neonate. Statistical significance was in fact higher for a greater number of frequencies in AS than QS. None-the-less, future studies may best be carried out with polysomnograms to determine sleep state, which may provide for more optimal sensitivity and specificity for this measure. Recordings in this study were also short, usually 10 minutes and only a short, initial sample of sleep was measured. Longer recordings may reveal even greater differences between HRA and LRC neonates.

The low coherence found at 2 and 4 Hz in the HRA cohort appears to be below what would be expected by volume conduction alone, suggesting that neonates at HRA as a group have little to no functional connectivity between the hemispheres in sleep, at least for Central and Occipital regions examined here. If substantiated in a larger study, this finding raises critical concerns about the impact on brain and behavioral development from impaired connectivity starting at such a young age and during sleep which is so critical for learning and memory (Wang 2011, Huber and Born 2014, Buckley 2015). Coherence abnormalities in sleep state have been examined in a very limited number of studies in ASD. In two studies, Lazar (2010) studying Aspergers in SWS sleep and Leveille (2010) studying ASD children in REM sleep, and no significant difference in inter-hemispheric coherence was found when compared to age-matched controls. Buckley et al (2015) studied 2-6 year olds with ASD, DD and typical development across awake, REM sleep and SW sleep states and found a dynamically evolving pattern of connectivity across ages; but in SWS only, they did find a significant increase in coherence and decrease in phase lag in frontal-parietal regions which correctly distinguished the majority of ASD children from the other two groups. This study's findings differs from results obtained herein, specifically in the nature of the abnormality in inter-hemispheric coherence. Although likely related to age, studying the sleep state may help explain differences in connectivity.

Only two studies appear to have examined connectivity in HRA infants. Righi et al. (2014) showed diminished intra-hemispheric coherence in 12-month olds with HRA which parsed, not only the high vs. low risk subjects, but also identified HRA subjects that went on to develop autism. They did not report inter-hemispheric differences, in accordance with aspects the present disclosure. Orekhova (2014) found increased alpha phase lag inter-hemispheric connectivity in 14 month olds HRA that developed autism.

By comparison to infants at HRA, preterm infants, who have been shown by some to be at increased risk of ASDs (Mahoney 2013), generally show increased coherence and decreased spectral power in sleep at post-conceptual term compared to healthy term infants as reported by Scher 1994. Scher (1996) later examined the healthy “term” cohort and former preterm infants and showed lower mental scores on the Bayley at 24 months were predicted by higher coherence and lower power in beta frequencies on EEG in the newborn period/post-natal term age for premature infants. While other clinical cohorts will be needed to test specificity for HRA relative to coherence and power, preterm infants do not appear to show a similar neurophysiology or outcome risk profile.

### Behavioral Outcome in High-Risk Infants

A higher rate of ASD than expected was found among the HRA cohort, four of the eleven in the cohort or 36% whereas the literature reports recurrence rates between 10-20% (Bolton 1994, Landa 2006, Ozonoff 2011). However, new estimates suggest a recurrence rate in siblings to be close to 25%, when one takes stoppage in to account [Wood 2015], a rate also reported by Landa (2012). The high rate of autism at outcome described herein appears to have been influenced by the female that was diagnosed with an ASD who is a dizygotic twin and has two older male siblings with ASD, increasing her risk [Werling 2015].

Interestingly, her male twin in the HRA cohort did not meet ASD criteria but showed a NS-BAP. In addition, one of the males who developed autism is the sibling of a female with severe autism, which also placed him at a higher risk, up to 44% in one study [Werling 2015]. Interval between pregnancies, which has recently been shown to increase ASD risk [Zerbo 2015], was not examined. However, the number of subjects developing autism appears to be within expected rate taking multiple factors into account. The HRA population was also skewed to more males than females but it is not clear that this has a significant impact on outcome rates. Herein a higher than expected rate of BAP was also found in the HRA cohort, namely 36%. Published rates for BAP in infant siblings are reported at 15% [Landa 2012], although rates up to 50% are reported for parents of children with ASD [Dawson 2007]. Again one male subject with BAP had increased risk due to having three affected sibling, one his female twin and the other two older male siblings. A second subject showing signs of a BAP was born just 13 months after his next oldest sibling, which may have raised his risk given recent study results [Zerbo 2015]. Of note, his diagnostic classification improved from ASD at 20 months to NS on ADOS at 30 month although PDD-BI at 30 months was still reporting atypical behaviors. Also, the PDD-BI was used as a source for behavioral profile seeking BAP in HRA infant siblings, a tool that is not designed or studied for this purpose, which may have affected rates described herein.

All three high-risk males that developed an ASD had regressions documented on ADOS between 20 and 30 months, two of which were reported clinically. The one ASD female with heavy genetic loading for ASD did not show regression between 20-30 months. Lord (2012) showed two modes for regression in toddlers on the ADOS, one between 10 and 20 month and one between 20 and 30 months with roughly one-third regressing between 18 and 36 months. Kalb 2010, in a retrospective study of 2700 subjects found regression in 44% with a mean age of regression 19.63 months, quite similar to the timing of regression in the present sample. Ozonoff (2010) reported social behavior declines in a sample of infants with ASD that started before 12 months on coded video and tended to stabilize by 36 months. But this type of slow decline may relate more to neurophysiological risk factors such as high signal to noise ratio, and differ in mechanism from regressions that are later and more acute and profound.

The cause of more acute regressions remains unclear. Neurophysiologic causes including clinical epilepsy syndromes, i.e. West Syndrome and Landau Kleffner Syndrome, and subclinical epilepsies (Buckley 2016), though the role of isolated spikes on EEG are still being debated (Tharp 2004, Ghacibeh 201.5.

Neuropathological studies have shown activation of the innate immune system in the brains of children with autism and some hypothesize that additional immune challenges may precipitate regression. The concept that a brain that is over active (High E/I ratio) (Rubinstein 2003) with higher energy demands has led some to hypothesize that substrate deficits with or without metabolic disorders might precipitate regressions (Rossignol 2014). The change in brain oscillatory patterns to higher gamma frequencies with greater metabolic demands from 16-24 months of age when regressions are commonly seen supports that argument. No cause for regression was apparent in subjects studied herein.

### Decreased EEG Coherence and Decreased Right Power in the Neonatal Period Predict Autism Diagnosis and Autistic Behavioral Severity at 30-Month Outcome

Correlation analyses of HRA neonatal sleep EEG and outcome measures at 30 months of age revealed strong correlations between EEG Coherence and Right Power and later ASD diagnosis/behavioral phenotype. Lower coherence predicted worse autistic symptomatology and diagnostic classification, and lower R power predicted worse autistic symptomatology. Specifically, it was found that the lower the functional connectivity between the hemispheres in early infancy as measured by Coherence at 4 Hz, the higher the grade of autism based on Best Estimate Diagnosis at outcome (CC: 0.72, p=0.013). This finding was supported by strong correlations between Coherence at 4 Hz and ADOS scores (Social Affect and RBB) and PDD-BI scores (Social Pragmatics, Arousal Regulation and Autism Composite). Coherences at higher frequencies also showed strong correlations, particularly around 14 and 16 Hz such that lower coherence predicted worse PDD-BI scores (RRB, SocPP, Arousal, SocApp, and Autism Composite), with highest significance for social approach. Interestingly, Power in the right hemisphere from 2 to 6 Hz was also strongly associated with autistic behaviors as measured by Social Affect scores on the ADOS, such that as power on the right decreased, social affect scores worsened (CC: −0.72, p=0.031 @ 4 Hz). Wang (2013) has hypothesized that low alpha power in ASD is due to a failure of GABA interneurons to support alpha oscillations. The diminished power at 4 Hz may be a reflection of insufficient GABA inhibition. The finding herein that low right hemispheric power in the newborn period can be predictive of worse social behavior at outcome was unexpected and has not to the knowledge of the inventors been reported previously. The high correlations between neonatal EEG coherence and R power and later autistic behavior severity provide evidence that these EEG signatures are neonatal biomarkers for autism. Interestingly, Left power, which parsed the HRA cohort from the LRCs in the newborn period, was not associated with autistic behaviors at outcome, other than behaviors in the language domain.

### Increased EEG Left Power and Decreased Coherence in the Neonatal Period Predict Language Impairments at 30-Month Outcome

Correlation analyses of neonatal Sleep EEG and HRA group outcome measures at 30 months of age revealed very strong correlations between Left Power and language measures at outcome. Increased left power was strongly correlated with poorer receptive and expressive language on multiple measures including the PDD-BI, MSEL and VABS. The most significant finding was for L-pwr and RL/EL/Verbal DQ on the MSEL (a direct assessment), which all showed strong correlations across essentially the entire power spectrum, 2-30 Hz. This finding was supported by strong L-Pwr correlations with Expressive language on the PDD-BI in the 2-8 Hz frequencies and with the Communication Domain on the VABS across the power spectrum. Decreased coherence in the newborn period was also somewhat predictive of poorer language at outcome though the correlations were far less impressive when compared to the correlations for L-pwr. Coherence was significantly correlated with the MSEL

Receptive language at 8 and 14 Hz and Verbal DQ at 14 Hz. The VABS did not provide any support for this finding and while there were significant correlations with the language composites on the PDD-BI, EXSCA-C and REXSCA-C, the Expressive language and LMRL domains showed no correlation with Coherence on the PDD-BI.

### L-R Hypoconnectivity in Autism

As a measure of functional connectivity, the reduced L-R coherence found in HRA neonates provides evidence that hypo-connectivity, well described in autistic children and adults using qEEG (Dawson 1982, Murias 2007, Coben 2008, Isler 2010, Duffy 2012, Coben 2013, Moseley 2015, Matlis 2015), and other methodologies MEG and fMRI [Mohammad-Rezazadeh 2016]), is already present in the neonatal period in infants who go on to develop ASD. Inter-hemispheric hypo-connectivity has not been described previously in HRA infants. Hyper-connectivity in frontal regions has been described in 14 month-old HRA infants that developed autism (Orekhova 2014). Further analysis of data may be performed to determine if the L-R hypo-connectivity found for Central-Occipital regions is occurring at the same time as frontal hyper-connectivity.

### Hemispheric Asymmetry—Increased Left Power in Autism and Language Disorders

Increased L-hemisphere power has been described in the autism literature from the earliest qEEG studies done in autistic children by Cantor et al. (1986) and more recently by Doust (2004) who found increased L frontal theta power in adults with ASD. Stroganova (2007) showed increased left power in delta, theta and alpha frequency bands in a cohort of autistic boys. Sutton (2005) and Burnette (2011) studied high functioning children with ASD (HFA) and showed increased left power was associated with less social impairment but increased social anxiety (Sutton 2005) and more impaired verbal abilities as measured by Verbal IQ (Burnette 2011). The Sutton and Burnette studies' results are similar to present findings where increased L power in the HRA neonates was less correlated with social impairment, yet predicted worse language and V-IQ at outcome.

EEG power in HRA has been examined in only two studies previously. Gabar-Durnam (2015) examined frontal power in HRA vs. LRCs seeking hemispheric asymmetry in the alpha band and found left power was significantly higher than right power in HRA 6 month olds; but absolute power was not presented so it is not clear that Left power was actually increased over controls. Tierney (2012) found decreased bifrontal power across the power spectrum in HRA compared to LRC with no hemispheric differences. Abnormal hemispheric specialization in autism has been described extensively in the literature. EEG/ERP and fMRI studies concur that responses to speech sound in the left hemisphere are diminished, are usually preserved on the right, and language is largely lateralized to the right with more mixed dominance in individuals with more highly developed language. (Dawson 1982, 1986, 1989), (Knaus 2010) (Eyler 2012) (Neilsen 2014). It is hypothesized that the increased power in the left hemisphere measured here and in other studies in ASD results in a low signal to noise ratio that undermines processing of simple speech sounds and higher language (Wang 2013) and drives language lateralization to the right hemisphere. Several studies have shown right lateralization of language in ASD using structural MRI (Herbert 2002) (Floris 2016) and DTI (Lange 2010) and multimodal methods (Berman 2016). Atypical hemispheric specialization for language in children with dysphasia (SLI) has been shown in the absence of ASD (Dawson 1989). Atypical hemispheric specialization for faces as measured by left lateralized intra-hemispheric coherence has been shown in HRA infants at 12 months of age, and the more lateralized to the left, the more likely the infant was to go on to develop ASD (Keehn 2015).

### Hemispheric Asymmetry—Decreased Right Power in Autism

In subjects studied herein, decreased power on the right was found to correlate with severity of autistic social behavior independent of Left power. While Leftward Hemispheric asymmetry suggests lower power on the right, this is not always the case and, in any case, is not occurring independent of left power. The finding of decreased right power in children or adults with ASD is not reported in the literature to the best knowledge of the inventors, and may be a novel feature peculiar to the neonatal period. Specifically, it appears to be a marker of infants at greatest risk of developing more severe forms of the disorder and thus may be a marker of a lack of ability of the right hemisphere to take over some functions of a noisy, poorly functioning left hemisphere.

### Increased EEG Left Power in the Neonatal Period Predicts Worse Adaptive and Motor Skills at 30 Months

An additional finding in the present study was the correlation of neonatal Left power with Adaptive and motor skills at outcome. Left power was negatively correlated with the Adaptive Behavior Composite at 2-6 Hz reflecting the sum of similar findings for the domains contributing to the ABC, Communication, DLS, Social and Motor skills all of which showed significant negative correlations. Left power was also negatively correlated with Fine and Gross Motor domains on the MSEL. By comparison, R power did not correlate with any domains on the VABS or MSEL. This finding of increased left power in HRA neonates predicting poorer adaptive and motor skills may well reflect similar mechanisms as discussed above for language: high left power likely impacts signal to noise ratio, information processing and hemispheric lateralization which could interfere with handedness/fine motor development, motor coordination and the daily living skills sets and behaviors which are dependent on motor competence.

### EEG Power and Coherence in the Neonatal Period Did NOT Predict Non-Verbal DQ at 30 Months

Notable was the absence of any significant correlation between Coherence, Left or Right power and the Non-verbal DQ or Visual recognition domain on the MSEL which stands in sharp contrast to the correlation results for Left power and Language and V-DQ. As non-verbal intelligence is well known to vary relatively independent of ASD severity and language development, the lack of correlation here provided some validation of the findings in other domains.

Predicting Developmental Outcome from EEG Signatures in the Neonatal Period

Given the rapidly changing brain during development from birth to the age range of behavioral manifestation of ASD, ˜12-24 months of age, findings in the neonatal period of decreased coherence, increased left power and decreased R power are to some degree surprising; but, if replicated in a larger HR and LR samples, appear to reveal that neural signatures described in studies of children diagnosed with ASD are already present in high-risk neonates, providing further evidence for the prenatal origins of ASD. The combination of three EEG signatures, low coherence, high left power and low right power constitutes the profile of an infant at the highest risk of developing autism, based on the data in the present small cohort. In the present findings, neonatal EEG signatures both parse HRA infants from LRC infants and predict autism and behavioral severity at 30-month outcome, if replicated, will motive further work to elucidate the best EEG biomarker for autism in the newborn period.

### Timing of Neurophysiologic Abnormalities in Autism: The Clock is Ticking at Birth

The neurophysiological abnormalities found in neonates who later develop ASD have been described extensively in the literature. Abnormalities of functional connectivity in autism are well described even though matters of nodes, networks and scale are still being elucidated (Kitzbichler 2015). Abnormalities of spectral power, signal to noise ratio and hemispheric specialization, how they are intertwined and change over development are areas of active investigation (Tordiman 2015). The timing of these neurophysiologic mechanisms, in particular their presence in the neonatal period is revealed by the findings of this study.

Interventional Strategies Existing and in Development that May Improve Developmental Outcome in Infant Showing ASD Markers in Infancy and the Argument for Treatment in Early Infancy

The need for a biomarker for ASDs at or shortly after birth is becoming increasingly clear as structural and functional impairment in infants later diagnosed with autism (IDA) are being identified at younger ages in infancy. Structural brain changes are already present by 12-15 months of age in IDA as evidenced by increased cerebral volume on MRI (Amaral 2013); and the IBIS Network, using MRI/DTI, has shown microstructural abnormalities in white matter tracts (Wolff 2012) and corpus callosum and increased corpus callosum area and thickness by 6 months of age in IDA (Wolff 2015). In fact an increasing body of work suggests that structural brain changes are present even before birth [Casanova 2002], [Wegiel 2010], [Stoner 2014]. Functional brain changes are also being shown at increasingly younger ages. Orehkova (2014) showed elevated phase lag alpha connectivity at 14 months in IDA and Righi (2014) using coherence calculations showed reduced intra-hemispheric connectivity by 12 months of age in IDA. Poor eye contact in children with ASD has motivated studies on eye gaze and abnormal neural responses to eye gaze shift by 6-10 months of age in IDA has been shown by the BASIS group (Elsabbaugh 2012); and Jones and Klin (Jones 2013) have shown that visual fixation to eyes which is normal at 2 months in IDA has already deteriorated by 6 months of age. Initiating treatment protocols before brain function declines in the first six months of life will be critical to achieving optimal outcome [Brian 2015, Klin 2015].

Interventional programming aimed to achieve this end, such as the Early Start Denver Model (ESDM) developed by Rogers and Dawson (Estes 2015), are already showing not only improved behavioral outcome, but also neurophysiologic processing by ERP to faces and objects (Dawson 2012). Earlier identification would permit trials of interventions even earlier in life. Medical intervention trials being undertaken in children and adults diagnosed with ASD are also showing promise in core features and are based on neuropathologic and metabolic actions, which on a theoretical basis have a rationale for use in the neonatal period [Cellot and Cherubini 2014], [Liu 2016], [Bruining 2015], [Tyzio 2014] [Penagarikano 2015] [Ben-Ari 2015], i.e. Bumetanide [Lemonnier 2010, 2012], [Holmes 2015] [Du 2015], Sulforaphane [Singh 2014, 2016], Oxytocin [Anagnostou 2014, Young and Barrett 2015] and, potentially, CRF modulators [Gao 2016] with the goal of improving brain health and function and later behavioral outcome.

Assuring accuracy of neonatal diagnosis through a brain-based biomarker would be critical before treatment trials in infants at the greatest risk of autism might be considered. Infant sibling of children with Autism, who are already at higher risk, who also show clearly abnormal and predictive neurophysiology at birth might be the best candidates for such interventional trials. The abnormal neural signatures in the neonatal EEGs of the present HRA cohort who went on to develop ASD suggest that finding an EEG algorithm of sufficient sensitivity and specificity for an accurate ASD diagnosis to permit and motivate interventional drug trials in the neonatal period is not only possible, but likely.

Establishing the safety of medical interventions in the newborn infant, especially interventions aimed to change brain development, pose practical and ethical challenges, especially when efficacy is yet to be established. Additional unresolved ethical issues around very early identification and treatment of infants destined to develop autism spectrum disorders range from the impact of very early diagnosis on parent-infant bonding, parental stress and parenting more generally, to issues around neurodiversity [Walsh 2011]. But the potential for prevention, at the very least, of the most severe forms of autism through the discovery of a neonatal biomarker must be weighed against these cautionary concerns (Szatmari 2016). The present work supports the notion that careful pursuit of a valid neonatal EEG biomarker has the potential to provide neonatal identification that can help direct therapy, and may be useful in monitoring natural history of brain development and efficacy of interventions to improve developmental outcome. Ultimately, the use of multiple biomarkers, including genomic, immune, and metabolomics markers (Goldani 2016) in combination with neurophysiologic biomarkers (Mohammad-Rezazadeh 2016), holds the greatest promise for early identification, individualization of treatment, and optimal outcome in individuals with autism spectrum disorders.

Finally, the present methodology, which employs only four leads to distinguish HRA vs. LRC infants and predicts ASD diagnosis at outcome, is well poised to be developed through further study as a neonatal screening tool. The brainstem auditory evoked response used ubiquitously to screen newborns for hearing impairment employs six leads and has proven to be very feasible and reliable. Thus the present approach that utilizes brain field potentials from four leads is likely to prove to be both feasible and reliable for detecting ASD using the present analytical methods, assuming results hold on a large scale and with the development of a referential database. It is noteworthy that EEG measurements of a patient can change very quickly in the first hours, days and weeks after birth. Thus, it remains to be seen whether infants in the first 24-48 hours of life, when newborn screenings typically are conducted in hospital, can retain the same or similar neural signatures of three week-old infants studied herein. Similarly, it remains to be determined how well the present EEG signatures hold up in premature infants or infants with concurrent medical conditions.

Infants with genetic conditions associated with autism, such as Angelman or Rett syndrome, may well have EEG signatures that differ from infants without recognizable genetic disorders. Further study of large populations of unselected newly born infants with long-term follow-up will be needed to ascertain the best algorithm for a neonatal diagnosis with sufficiently high sensitivity and specificity as to meet the standard for a newborn screening test in the general population. The findings of this study, however, if replicated, provide evidence that an EEG biomarker in the neonatal period exists and suggests that a biomarker at birth is likely to exist. Further studies of EEG biomarkers in the neonatal period are warranted and use of such a neonatal EEG biomarker as a newborn screening test for ASD is promising.

### Ontogeny of Autism and Related Disorders: Excessive GABA Excitation Prenatally, Delayed GABA Switch, and Neonatal EEG Finding in Infants at High-Risk of ASD.

The brain is abnormal in ASD before birth. Structural alterations emanating from the prenatal period have been confirmed. That functional abnormalities are present at or soon after birth has now been confirmed by the present work. These findings, though based on a small sample and in need of replication, are the first to provide evidence that abnormal brain neurophysiology in a neonate who will become autistic is present at or soon after birth. While the mechanisms by which these neurophysiologic derangements develop are not yet understood, recent hypotheses bear mentioning. Numerous studies using a variety of methods are confirming abnormalities in GABAergic mechanisms in autism. Delays in the switch from excitatory to inhibitory GABAergic mechanisms during gestation and postnatally may contribute substantially to alter very early ontogenic mechanisms, to drive up the E/I ratio (Rubenstein 2003), to change forever the careful orchestration of building a brain: its structure, its set points, and its rhythms. Alterations in power and coherence are potential manifestations of and likely stem from these mechanisms. Metabolic demands created by these brain changes may not always be met in the developing brain, before and/or after birth. Immune mechanisms involving the maternal-fetal unit may play a key role in driving early GABAergic mechanisms; and interplay between the environment and genome may well be playing a substantial role in altering the very early immune balance being struck between a mother and her fetus.

The vulnerability of the L hemisphere in autism is likely related to the evolutionary brain changes that occurred as primates evolved to use tools and hominids evolved to use language. The vulnerability of the limbic system and the social brain may well stem from far earlier branches in the evolutionary tree when older, deeper structures related more closely to mammalian survival developed to make us more social beings. It is likely that these evolutionary vulnerabilities underlie the phenotype of autism, as it is these evolutionary changes that made us human.

The present invention has been described in terms of one or more preferred embodiments, and it should be appreciated that many equivalents, alternatives, variations, and modifications, aside from those expressly stated, are possible and within the scope of the invention.

1. A method for determining a risk for a neonatal patient to develop an autism spectrum disorder (“ASD”), the method comprising:
coupling a sensor assembly comprising plurality of electroencephalogram (“EEG”) sensors to a neonatal patient;
acquiring, using the sensor assembly, EEG data during a sleep state of the neonatal patient;
analyzing the EEG data to determine neural signatures indicative of a brain activity of the neonatal patient during the sleep state;
generating, based on the neural signatures, a composite representing a neurofunctional profile of the neonatal patient;
determining a risk for the neonatal patient to develop an autism spectrum disorder (“ASD”) by comparing the composite to a reference; and
generating a report indicating the risk.

2. The method of claim 1, wherein the method further comprises computing, using the EEG data, power spectra associated with different locations about the neonatal patient's head.

3. The method of claim 2, wherein the different locations include a right brain hemisphere and a left brain hemisphere of the neonatal patient.

4. The method of claim 3, wherein the method further comprises computing a difference of spectral power between the right brain hemisphere and the left brain hemisphere.

5. The method of claim 1, wherein the method further comprises computing a coherence between a right brain hemisphere and a left brain hemisphere of the neonatal patient.

6. The method of claim 1, wherein the neural signatures are computed using at least one of a spectral information, a power information, a coherence information, a phase information, a synchrony information, and an asymmetry information.

7. The method of claim 1, wherein an age of the neonatal patient is less than approximately 1 month.

8. The method of claim 1, wherein the composite is generated based on a weighted combination of different neural signatures.

9. The method of claim 1, wherein determining the risk includes utilizing at least one characteristic of the neonatal patient.

10. A method for determining a likelihood for a neonatal patient to develop a neurobehavioral disease, the method comprising:
receiving electroencephalogram (“EEG”) data acquired from a neonatal patient during a sleep state;
generating at least one of a spectral power and coherence information using the EEG data;
assembling a neurofunctional profile of the neonatal patient using the at least one of spectral power and coherence information;
correlating the neurofunctional profile with a reference to determine a likelihood for the neonatal patient to develop a neurobehavioral disease; and
generating a report using the likelihood.

11. The method of claim 10, wherein the method further comprises computing, using the EEG data, spectral power associated with different locations about the neonatal patient's head.

12. The method of claim 11, wherein the different locations include a right brain hemisphere and a left brain hemisphere of the neonatal patient.

13. The method of claim 12, wherein the method further comprises computing a difference of spectral power between the right brain hemisphere and the left brain hemisphere.

14. The method of claim 10, wherein the method further comprises computing a coherence between various portions of a right brain hemisphere and a left brain hemisphere of the neonatal patient.

15. The method of claim 10, wherein the method further comprises computing neural signatures using at least one of the spectral power and coherence information to assemble the neurofunctional profile.

16. The method of claim 10, wherein an age of the neonatal patient is less than approximately 1 month.

17. The method of claim 10, wherein the neural profile is generated based on a weighted combination of different neural signatures.

18. The method of claim 10, wherein determining the likelihood includes performing a statistical analysis utilizing at least one characteristic of the neonatal patient.

19. The method of claim 10, wherein determining the likelihood further comprises comparing coherence at multiple frequencies for different locations about the neonatal patient's head.

20. The method of claim 19, wherein the method further comprises comparing coherence values at low frequencies with coherence values at high frequencies.
